(19) United States
(12) Patent Application Publication
(10) Pub. No.: US 2017/0306001 A1
(43) Pub. Date: Oct. 26, 2017

(54) CHIMERIZATION AND CHARACTERIZATION OF A MONOCLONAL ANTIBODY WITH POTENT NEUTRALIZING ACTIVITY ACROSS MULTIPLE INFLUENZA A H5N1 CLADES

(71) Applicant: NATIONAL UNIVERSITY OF SINGAPORE, SINGAPORE (SG)

(72) Inventors: YEE JOO TAN, SINGAPORE (SG); TZE MINN MAK, SINGAPORE (SG)

(73) Assignee: NATIONAL UNIVERSITY OF SINGAPORE, SINGAPORE (SG)

(21) Appl. No.: 15/129,765

(22) PCT Filed: Mar. 27, 2015
(86) PCT No.: PCT/SG2015/000101

§ 371 Date: Sep. 27, 2016

Related U.S. Application Data
(60) Provisional application No. 61/971,268, filed on Mar. 27, 2014.

(51) Int. Cl. C07K 16/10; A61K 47/68; A61K 39/00

(52) U.S. Cl. CPC C07K 16/1018; A61K 47/6841; C07K 2317/76; C07K 2317/24; A61K 2039/507; C07K 2317/94; C07K 2317/34

(57) ABSTRACT

MAb 9F4 provides heterologous protection against multiple influenza A H5N1 clade viruses, including one of the recently designated subclades, namely 2.3.4, through binding to a novel epitope. The present invention relates to isolated mouse-human chimeric (xi) IgG₁-9F4 and IgA₁-9F4 MAb which retain high degrees of binding and neutralizing activity against influenza H5N1. The invention also relates to methods of production, kits and uses of the chimeric antibodies in the treatment of influenza A subtype H5N1 disease.

### FIELD OF THE INVENTION

The present invention relates to isolated mouse-human chimeric antibodies which retain high degrees of binding and neutralizing activity against multiple influenza A H5N1 clade viruses that infect humans, through binding to a novel conformational epitope, methods for producing same and uses thereof in treating human influenza infection.

### SEQUENCE LISTING

The present application contains a Sequence Listing which has been submitted electronically in ASCII format. The Sequence Listing is provided as a file entitled 169548_010200_Repl_SeqList.txt created Apr. 7, 2017, which is 9.45 kb in size. The information in the electronic format of the sequence listing is incorporated herein by reference in its entirety.

### BACKGROUND OF THE INVENTION

Highly pathogenic avian influenza A (HPAI) virus H5N1 remains a serious threat to global health due to its unabated and widespread geographical circulation. Although human cases remain sporadic, the absence of human herd immunity, the high lethality and potential ability of HPAI H5N1 to gain efficient human transmissibility, all point towards a potentially catastrophic pandemic.

The establishment and continual antigenic drift of H5N1 viruses in poultry and wild bird populations has led to the evolution of diverse lineages with distinct geographical distribution. This ongoing evolution of H5N1 viruses hampers vaccine development and enables emerging resistance to both adamantanes and neuraminidase inhibitors (Chao et al., 2012; Le et al., 2005). The increased usage of antiviral drugs may also contribute to the development of resistance (Tang et al., 2008). As such, there is a strong urgency for alternative strategies to be developed. Since antibodies are crucial in the protection against infection, passive immunotherapy is increasingly being explored as a viable option [reviewed in (Ye et al., 2012)]. The effectiveness of this approach has been documented for treatment of severe influenza illness during the 1918 pandemic influenza and, more encouragingly, H5N1 virus infected patients. Furthermore, several preclinical studies demonstrate the protective ability of neutralizing monoclonal antibodies (MAbs) against lethal H5N1 challenge (Du et al., 2013; Meng et al., 2013; Ye et al., 2010). The use of MAb is advantageous over traditional convalescent blood products in terms of availability and inter-batch consistency.

Due to its abundance and role in virus entry, the surface hemagglutinin (HA) glycoprotein elicits the production of neutralizing antibodies and this forms the basis of conventional vaccination and most passive immunotherapeutic strategies. These antibodies confer protection against infection as they block viral entry into host cells by interfering with virus attachment or by preventing HA-mediated membrane fusion during virus uncoating.

However, because influenza replication is error prone, selection of escape mutants may occur if strategies are based on single MAb formulations. To overcome this problem, a combination of non-competing MAbs can be used synergistically to confer broad protection while preventing emergence of escape variants. Proof of this concept has been shown for several respiratory viruses, including HPAI H5N1 (Prabakaran et al., 2009; Ter Meulen et al., 2006). Thus, studies involving the pre-pandemic generation and epitope mapping of neutralizing MAbs would collectively aid in facilitating rapid accessibility to and selection of the appropriate combination of MAbs for passive immunotherapy in the event of a pandemic.

Although fully human anti-H5N1 HA neutralizing MAbs have been described, the generation of such antibodies typically require H5N1 convalescent donors as cross-protective antibodies obtained from patients previously immunized with other subtypes of influenza are rare. Consequently, the mouse hybridoma technology continues to be a popular method for the in vitro generation of anti-H5N1 HA MAbs. However, murine antibodies will elicit a non-self immune response in humans, rendering them useless or even harmful if used directly for immunotherapy. A solution is to make mouse-human chimeric constructs, consisting of the original mouse variable antibody domains fused to human constant domains. The resultant chimeric (xi-) MAb should retain the binding properties of the original mouse MAb, but with reduced immunotoxicity.

MAb 9F4 is a mouse IgG_{2b} antibody with neutralizing activity against multiple H5N1 viruses and recognizes a novel epitope (²⁶⁰I/LVKK²⁶³, according to H3 numbering) (Oh et al., 2010) that is situated away from previously characterized antigenic sites on HA globular head (Underwood, 1982; Wiley et al., 1981), suggesting that MAb 9F4 may be used in synergy with other characterized MAbs. In this study, we tested the ability of MAb 9F4 to bind HA of one of the recently designated subclades, namely 2.3.4, of H5N1 and extended the antigenic characterization of the MAb. Because of its potent neutralizing activity across multiple H5N1 clades and subclades, two chimeric (xi-) versions of the MAb, xi-IgG₁-9F4 and xi-IgA₁-9F4, were generated and tested to assess their therapeutic potential.

### SUMMARY OF THE INVENTION

The present invention provides neutralizing chimeric antibodies which specifically recognize a conformational (non-linear) epitope on influenza A H5N1 clades.

Accordingly, in a first aspect, there is provided an isolated chimeric antibody, variant, mutant or fragment thereof, wherein the antibody, variant, mutant or fragment thereof is capable of specifically binding to a conformational (non-linear) epitope of influenza A virus subtype H5N1, wherein the conformational epitope comprises the amino acid sequence ²⁶⁰I/LVKK²⁶³ (H3 numbering).

In a preferred embodiment of the disclosure, the conformational epitope comprises three antigenic sites, wherein a first site comprises the amino acid sequence I/LVKK, a second site comprises the amino acid sequence WLL and the third site comprises the amino acid sequence EWSYIV.

Another preferred embodiment of the disclosure relates to the antibody or fragment thereof being a humanized antibody.

In another preferred embodiment, the antibody comprises the mouse VH domain ligated to human IgG₁ heavy chain constant (CH) domain and the mouse VL domain ligated to human light chain kappa constant (CL) domain; or wherein the antibody comprises the mouse VH domain ligated to human IgA₁ heavy chain constant (CH) domain and the mouse VL domain ligated to human light chain kappa constant (CL) domain.

More preferably, the chimeric antibody comprises:
(a) a variable heavy chain comprising the amino acid sequence of SEQ ID NO: 1, a variant, mutant or fragment thereof, and a variable light chain comprising the amino acid sequence of SEQ ID NO: 2, a variant, mutant or fragment thereof, for mouse-human chimeric IgG₁ antibody, or
(b) a variable heavy chain comprising the amino acid sequence of SEQ ID NO: 5, a variant, mutant or fragment thereof, and a variable light chain comprising the amino acid sequence of SEQ ID NO: 6, a variant, mutant or fragment thereof, for mouse-human chimeric IgA₁ antibody.

In another preferred embodiment, the chimeric antibody has the H5N1 binding and neutralization characteristics of mouse monoclonal antibody 9F4.

In another preferred embodiment, the chimeric antibody binds to clade 2.3.4 H5N1 HA.

In another preferred embodiment, the antibody is linked with at least one drug, preferably an anti-viral drug.

In another aspect of the disclosure, there is provided a method of producing at least one mouse-human chimeric antibody which binds influenza A virus subtype H5N1, the method comprising the steps of:
a. Ligating a 9F4 VH domain nucleic acid encoding SEQ ID NO: 1 to a human IgG₁ CH domain and ligating a 9F4 VL domain nucleic acid encoding SEQ ID NO: 2 to a human CL domain in a single IgG₁ constant region expression vector; or
b. Ligating a 9F4 VH domain nucleic acid encoding SEQ ID NO: 5 to human IgA₁ CH domain in a first cloning vector, and ligating a 9F4 VL domain nucleic acid encoding SEQ ID NO: 6 to a human CL domain in a second cloning vector;
c. Transfecting the resulting chimeric construct or constructs into a suitable cell line; and
d. Collecting cell culture supernatants and extracting and purifying the chimeric antibody.

In another aspect of the disclosure, there is provided an isolated chimeric antibody produced according to the methods described herein.

In another aspect of the disclosure, there is provided the herein described chimeric antibody or a fragment thereof for use in medicine.

In another aspect of the disclosure, there is provided a method of treatment of influenza A subtype H5N1 disease, the method comprising administering to a subject in need thereof an efficacious amount of at least one chimeric antibody, variant, mutant or a fragment thereof according to the invention.

In another aspect of the disclosure there is provided the use of the antibody or a fragment thereof according to the invention for the preparation of a medicament for the treatment of influenza A subtype H5N1 disease.

In another aspect of the disclosure there is provided a kit for treating influenza A subtype H5N1 disease, the kit comprising at least one chimeric antibody or a fragment thereof according to the invention.

In another preferred embodiment there is provided an isolated nucleic acid molecule encoding:
(a) at least one variable heavy chain of the antibody or a fragment thereof according to the invention, wherein the heavy chain comprises the amino acid sequence of SEQ ID NO: 1, a variant, mutant or fragment thereof; and at least one variable light chain of the antibody or a fragment thereof according to the invention, wherein the light chain comprises the amino acid sequence of SEQ ID NO: 2, a variant, mutant or fragment thereof, or
(b) at least one variable heavy chain of the antibody or a fragment thereof according to the invention, wherein the heavy chain comprises the amino acid sequence of SEQ ID NO: 5, a variant, mutant or fragment thereof; and at least one variable light chain of the antibody or a fragment thereof according to the invention, wherein the light chain comprises the amino acid sequence of SEQ ID NO: 6, a variant, mutant or fragment thereof, and
   wherein the nucleic acid molecule encodes an IgG₁ or an IgA₁ chimeric antibody, respectively.

In another aspect of the invention, there is provided an expression vector comprising the chimeric nucleic acid molecule according to the invention.

In another aspect of the invention, there is provided a host cell comprising the expression vector as described above.

### BRIEF DESCRIPTION OF THE FIGURES

FIG. 1: MAb 9F4 binds and prevents viral entry into MDCK cells mediated by HA of clade 2.3.4 H5N1.

(A) Alignment of residues 229 to 288 (based on H3 numbering) in the HA protein of a clade 2.3.4 H5N1 virus (DL06) with the corresponding domain in two clade 1 viruses (Hatay04 and VN04). An epitope within the HA1 subunit previously found to be essential for the interaction with MAb 9F4, which was generated using Hatay04-HA, is boxed. For comparison, the mature H5 numbering is also included in this diagram.
(B) MDCK cells were transfected with empty vector or DL06-HA or Hatay04-HA expressing plasmids. Binding of 9F4 to surface expressed recombinant HAs was detected via immunofluorescence assay performed on non-permeabilized cells. Cells were stained with MAb 9F4 followed by Alexa Fluor® 488-conjugated goat anti-mouse IgG antibody. Hatay04-HA (clade 1) transfected cells were included as a positive control. Original magnification ×10.
(C) Pseudotyped lentiviral particles harboring the HA proteins from H5N1 influenza viruses of clade 1 (VN04-HApp) and clade 2.3.4 (DL06-HApp) were incubated with different concentrations of MAb 9F4 for 1 h before inoculation onto MDCK cells. Luciferase activity in the cell lysates was determined 72 h post-infection. Viral entry, as indicated by the luciferase activity measured in relative light units (RLU), was expressed as a percentage of the reading obtained in the absence of antibody, which was set at 100%. A control MAb 8F8 of the same isotype was used at 10,000 ng/ml. The experiments were repeated three times, and representative data are shown. Each histogram shows the mean of the values from duplicate wells. Error bars, standard deviation. Inset dotted lines demarcate approximate IC₅₀.

FIG. 2: xi-IgG₁-9F4 retains binding and neutralization ability against multiple H5N1 clades.

(A) MDCK cells were transfected with empty vector or the various H5-HA expressing plasmids. Binding of xi-IgG₁-9F4 to surface expressed recombinant HAs was detected via immunofluorescence assay performed on non-permeabilized cells. Cells were stained with xi-IgG₁-9F4 followed by Alexa Fluor® 488-conjugated goat anti-human IgG antibody. Original magnification ×40.
(B-E) Pseudotyped lentiviral particles harbouring the HA proteins from the various representative clade H5N1 influenza viruses were incubated with different concentrations of MAb 9F4 or xi-IgG₁-9F4 for 1 h before inoculation onto MDCK cells. Luciferase activity in the cell lysates was determined 72 h post infection. Viral entry, as indicated by the luciferase activity measured RLU, was expressed as a percentage of the reading obtained in the absence of antibody, which was set at 100%. A control MAb 8F8 of the same isotype was used at 10,000 ng/ml. The experiments were repeated three times, each in duplicates. Each histogram shows the mean of the values from all data. Error bars, standard deviations. Differences in binding by mouse and xi-IgG₁-9F4 were evaluated by unpaired t-test (* p<0.05, ** p<0.01). Inset dotted lines demarcate approximate IC₅₀.

FIG. 3: Neutralization ability of xi-IgA₁-9F4 is reduced due to decreased binding affinity.

(A) Pseudotyped lentiviral particles harboring the HA proteins from VN04 were incubated with different concentrations of MAb 9F4 or xi-IgA₁-9F4 for 1 h before inoculation onto MDCK cells. Luciferase activity in the cell lysates was determined 72 h postinfection. Viral entry, as indicated by the luciferase activity measured RLU, was expressed as a percentage of the reading obtained in the absence of antibody, which was set at 100%. MAb 8F8 was used at 10,000 ng/ml as a negative control MAb. The experiments were repeated three times, each in duplicates. Each histogram shows the mean of the values from all data. Error bars, standard deviations. Differences in binding by mouse and xi-IgA₁-9F4 were evaluated by unpaired t-test (* p<0.05, ** p<0.01). Inset dotted lines demarcate approximate IC₅₀.

(B-D) Comparative ELISA as performed to measure the binding of different forms of MAb 9F4 to fixed amount of cell lysates obtained from cells transfected with a cDNA construct expressing various H5 HA. All readings are normalized against cell lysates from 293FT cells transfected with empty vector alone. The experiments were repeated three times. Each point shows the mean of the values from all data. Error bars, standard deviations. The cut-off level was determined using a control mouse MAb.

FIG. 4: Mouse and mouse-human chimeric forms of MAb 9F4 comparably inhibit HA mediated fusion at low pH.

HeLa cells were transiently transfected with a cDNA construct expressing Hatay04-HA and then incubated with mouse-9F4 or xi-IgG₁-9F4 at two different concentrations. Control cells were not treated or incubated with control mouse-8F8 antibody. Subsequently, the unbound MAbs were removed by washing the cells with 1×PBS prior to treatment with low pH buffer and followed by recovery, fixation and staining. Plasma membrane is stained orange (CellMask Orange) and nucleus is stained blue (DAPI). Pictures shown are representative of 20 fields and 3 independent experiments. The top two panels were taken at original magnification ×10 while the bottom panel was taken at original magnification ×40.

FIG. 5: MAb 9F4 recognizes a conformation dependent epitope.

(A) ELISA was performed to measure the binding of MAb 9F4 to recombinant and purified HA1 protein (Nchicken/India/NIV33487/2006(H5N1)) and ²⁵⁹KIVKKGDSTIM²⁶⁸ (based on H3 numbering) (SEQ ID NO: 21) synthetic peptide. All the residues in the peptide are present in the HA1 protein. Each histogram shows the mean of the values from duplicate wells. Error bars, standard deviations.
(B) Lysates of 293FT cells expressing HA of different clades of H5N1 were used in western blot analysis. One set of samples was prepared in Laemmli sample buffer containing DDT and boiled to yield completely reduced and denatured HA and then analyzed using MAb 9F4 (middle panel). Another set of samples was prepared in Laemmli sample buffer containing DDT but not boiled to yield partially denatured HA (right panel). Expression levels of HA proteins were checked using a rabbit polyclonal antibody raised against the N terminus of HA (top left panel) and levels of endogenous actin levels were checked as loading control (all bottom panels).

FIG. 6: Additional residues upstream of ²⁶⁰I/LVKK²⁶³ in the HA1 protein are required for the interaction with MAb 9F4 but deglycosylation does not affect binding. H3 numbering is used in this figure.

(A) Schematic representation of the HA constructs used for epitope mapping according to H3 numbering. −16 to 550aa depicts the full length Hatay04-HA protein and black box represents ²⁶⁰I/LVKK²⁶³, which were previously shown to be essential for the interaction with 9F4. SP at N-terminus indicates signal peptide.
(B) Full length and truncated Hatay04-HA were expressed in 293FT cells and subjected to western blot analysis (without boiling) with MAb 9F4 (top panel). Polyclonal rabbit anti-HA antibody (bottom panel) was also used to check the expression of mutants.
(C) The fragment corresponding to −16 to 289aa of Hatay04-HA was untreated or treated with endoglycosidase H (Endo H), which removed the N-linked oligosaccharides from HA and caused a reduction in molecular weight, and then subjected to Western blot analysis (without boiling) with MAb 9F4.

FIG. 7: Sequence and annotation of the immunoglobulin genes of MAb 9F4.

The sequences of the A) VH and B) VL domains were obtained by RT-PCR performed on RNA extracted from the MAb 9F4 hybridoma. Sequences in bold, underlined or highlighted in grey represent variable (V) region, diversity (D) and joining (J) regions respectively. These highlighted segments contain complementarity determining region (CDR) 1-3 and were cloned into vectors containing human heavy and light constant domains to form chimeric MAbs.

FIG. 8: Predicted 9F4 epitopes

(A) Sequence alignment of −16-286aa of Hatay04, VN04, NethH7 and HKH9. The numbering convention used is based on mature H5. Epitopes were predicted by either BPAP or BEPro and were selected for testing based on conservation within H5 HA but not H7 and H9 HA (underlined sequences between aa60-80). The previously identified epitope is underlined at aa256-259 (based on H5 numbering). “*” denotes amino acid conservation, “.” denotes semi-conserved substitutions, “:” denotes amino acid substitution within the same amino acid group.

FIG. 9: Predicted epitopes spanning aa60-62 and aa75-80 (based on H5 numbering) are essential for 9F4 binding. (A) Schematic of triple alanine mutants tested. (B) Wild-type and mutant Hatay04 were screened against 9F4 in immunofluorescence assay. The gene segments coding for the different mutants were generated by PCR and cloned into PXJ3′ vector and expressed in MDCK cells. Binding by 9F4 or Rb anti HA(N) was detected by Alexa Fluor® 488-conjugated secondary antibodies.

FIG. 10: Effect of mutation on HApp binding. Equal amounts of HApp (based on p24 titre) expressing the wild-type and mutant Hatay04 were coated onto 96-well plates and detected using 1 μg/ml 9F4. Incorporation of wild-type and mutant Hatay04 into HApp was checked using Rb anti HA(N). Results are normalized against pseudotyped particles devoid of HA. Histogram and error bars represent mean and SD of triplicate wells.

### DETAILED DESCRIPTION OF THE INVENTION

### Definitions

Certain terms employed in the specification, examples and appended claims are collected here for convenience.

The terms “amino acid” or “amino acid sequence,” as used herein, refer to an oligopeptide, peptide, polypeptide, or protein sequence, or a fragment of any of these, and to naturally occurring or synthetic molecules. Where “amino acid sequence” is recited herein to refer to an amino acid sequence of a naturally occurring protein molecule, “amino acid sequence” and like terms are not meant to limit the amino acid sequence to the complete native amino acid sequence associated with the recited protein molecule.

As used herein, the term “antibody” refers to any immunoglobulin or intact molecule as well as to fragments thereof that bind to a specific epitope. Such antibodies include, but are not limited to polyclonal, monoclonal, chimeric, humanised, single chain, single chain fragment variable (scFv), Fab, Fab′, F(ab)′ fragments and/or F(v) portions of the whole antibody. The term “monoclonal antibody” may be referred to as “Mab”. The antibody includes antibodies xi-IgG₁-9F4 and xi-IgA₁-9F4, produced according to the invention. The antibodies, xi-IgG₁-9F4 and xi-IgA₁-9F4 may be monoclonal antibodies, polyclonal antibodies, single-chain antibodies, and fragments thereof which retain the antigen binding function of the parent antibody. The antibodies xi-IgG₁-9F4 and xi-IgA₁-9F4 are capable of specifically binding to influenza A subtype H5N1, including a conformational epitope comprising the amino acid sequence I/LVKK (SEQ ID NO: 17; SEQ ID NO: 18), the amino acid sequence WLL (SEQ ID NO: 19) and the amino acid sequence EWSYIV (SEQ ID NO: 20) or variants thereof that do not significantly reduce the antigenicity of the epitope and include monoclonal antibodies, polyclonal antibodies, single-chain antibodies, and fragments thereof which retain the antigen binding function of the parent antibody.

The term “antibody fragment” as used herein refers to an incomplete or isolated portion of the full sequence of the antibody which retains the antigen binding function of the parent antibody. Examples of antibody fragments include scFv, Fab, Fab′, F(ab′)2, and Fv fragments; diabodies; linear antibodies; single-chain antibody molecules; and multi-specific antibodies formed from antibody fragments. Fragments of the xi-IgG₁-9F4 and xi-IgA₁-9F4 antibodies are encompassed by the invention so long as they retain the desired affinity of the full-length antibody. In particular, it may be shorter by at least one amino acid. A single chain antibody may, for example, have a conformation comprising SEQ ID NO: 1 and SEQ ID NO: 2 with a linker positioned between them.

The term “chimeric antibody,” as used herein, refers to at least one antibody molecule in which the amino acid sequence in the constant regions has been altered so that the antibody more closely resembles a human antibody, and still retains its original binding ability.

The term “humanized antibody”, as used herein, refers to at least one antibody molecule in which the amino acid sequence within the variable and constant regions has been altered so that the antibody more closely resembles a human antibody, and still retains its original binding ability.

As used herein, the term “hybridoma” refers to cells that have been engineered to produce a desired antibody in large amounts. For example, to produce at least one hybridoma, B cells are removed from the spleen of an animal that has been challenged with the relevant antigen and fused with at least one immortalized cell. This fusion is performed by making the cell membranes more permeable. The fused hybrid cells (called hybridomas), will multiply rapidly and indefinitely and will produce at least one antibody. An example of a hybridoma is the cell line 9F4.

The term “immunological binding characteristics” of an antibody or related binding protein, in all of its grammatical forms, refers to the specificity, affinity and cross-reactivity of the antibody or binding protein for its antigen.

The term “isolated” is herein defined as a biological component (such as a nucleic acid, peptide or protein) that has been substantially separated, produced apart from, or purified away from other biological components in the cell of the organism in which the component naturally occurs, i.e., other chromosomal and extra-chromosomal DNA and RNA, and proteins. Nucleic acids, peptides and proteins which have been isolated thus include nucleic acids and proteins purified by standard purification methods. The term also embraces nucleic acids, peptides and proteins prepared by recombinant expression in a host cell as well as chemically synthesized nucleic acids.

The term “neutralising antibody” is herein defined as an antibody that can neutralise the ability of that pathogen to initiate and/or perpetuate an infection in a host. The invention provides at least one neutralising chimeric monoclonal antibody, wherein the antibody recognises an antigen from influenza A subtype H5N1.

The term “mutant” is herein defined as one which has at least one nucleotide sequence that varies from a reference sequence via substitution, deletion or addition of at least one nucleic acid, but encodes an amino acid sequence that retains the ability to recognize and bind the same conformational epitope on influenza A virus subtype H5N1 as the un-mutated sequence encodes. The term ‘mutant’ also applies to an amino acid sequence that varies from at least one reference sequence via substitution, deletion or addition of at least one amino acid, but retains the ability to recognize and bind the same conformational epitope on influenza A virus subtype H5N1 as the un-mutated sequence. In particular, the mutants may be naturally occurring or may be recombinantly or synthetically produced. More in particular, the mutant may be of at least 50%, at least 60%, at least 70%, at least 80%, at least 90%, or at least 95% sequence identity to the reference sequences. For example, the xi-IgG₁-9F4 variable light chain amino acid sequence set forth in SEQ ID NO: 2 is shorter than the full sequence set forth in Table 1 and may be considered a mutant of the full sequence in Table 1 because it retains the ability to recognize and bind the same conformational epitope on influenza A virus subtype H5N1.

The term “primers,” as used herein, refers to a nucleic acid sequence of at least about 6 nucleotides to 60 nucleotides, preferably about 15 to 30 nucleotides, and most preferably about 20 to 25 nucleotides, which can be used in PCR amplification or in a hybridization assay. As used herein, the term “oligonucleotide” is substantially equivalent to the terms “amplimers, “oligonucleotides”, “oligomers” and “probes,” as these terms are commonly defined in the art.

The term “sample,” as used herein, is used in its broadest sense. A biological sample suspected of containing nucleic acids encoding at least one influenza A subtype H5N1 derived peptide, or fragments thereof, or influenza A subtype H5N1 itself may comprise a bodily fluid, an extract from a cell, chromosome, organelle, or membrane isolated from a cell, a cell; genomic DNA, RNA, or cDNA (in solution or bound to a solid support), a tissue, a tissue print and the like.

As used herein, the terms “specific binding” or “specifically binding” refer to that interaction between a protein or peptide and an agonist, an antibody, or an antagonist. The interaction is dependent upon the presence of a particular structure of the protein recognized by the binding molecule (i.e., the antigenic determinant or epitope). For example, if an antibody is specific for epitope “A,” the presence of a polypeptide containing the epitope A, or the presence of free unlabeled A, in a reaction containing free labeled A and the antibody will reduce the amount of labeled A that binds to the antibody.

The term “subject” is herein defined as vertebrate, particularly mammal, more particularly human. For purposes of research, the subject may particularly be at least one animal model, e.g., a mouse, rat and the like. In particular, for treatment of influenza A subtype H5N1 infection and/or influenza A subtype H5N1-linked diseases, the subject may be a human infected by influenza A subtype H5N1.

The term “treatment”, as used in the context of the invention refers to prophylactic, ameliorating, therapeutic or curative treatment.

The term “variant” as used herein, refers to an amino acid sequence that is altered by one or more amino acids, but retains the ability to recognize and bind the same conformational epitope on influenza A subtype H5N1 as the non-variant reference sequence. The variant may have “conservative” changes, wherein a substituted amino acid has similar structural or chemical properties (e.g., replacement of leucine with isoleucine). More rarely, a variant may have “non-conservative” changes (e.g., replacement of glycine with tryptophan). Analogous minor variations may also include amino acid deletions or insertions, or both. Guidance in determining which amino acid residues may be substituted, inserted, or deleted without abolishing biological or immunological activity may be found using computer programs well known in the art, for example, DNASTAR® software (DNASTAR, Inc. Madison, Wis., USA). For example, the xi-IgG₁-9F4 variable light chain amino acid sequence set forth in SEQ ID NO: 2 is shorter than the full sequence set forth in Table 1 and may be considered a mutant of the full sequence in Table 1 because it retains the ability to recognize and bind the same conformational epitope on influenza A virus subtype H5N1.

The term ‘variant’ is intended to also describe variations to the amino acid sequence of the influenza A virus subtype H5N1 conformational epitope comprising the amino acid sequence I/LVKK (SEQ ID NO: 17; SEQ ID NO: 18), the amino acid sequence WLL (SEQ ID NO: 19) and the amino acid sequence EWSYIV (SEQ ID NO: 20) that do not significantly reduce the antigenicity of the epitope in terms of eliciting antibodies which bind to and inhibit influenza A subtype H5N1 virus activity. Variants include conservative amino acid substitutions.

### ABBREVIATIONS

HA Hemagglutinin
HApp Lentiviral pseudotyped particles
HPAI Highly pathogenic avian influenza
MAb Monoclonal antibodies
xi Chimeric

The term “comprising” as used in the context of the invention refers to where the various components, ingredients, or steps, can be conjointly employed in practicing the present invention. Accordingly, the term “comprising” encompasses the more restrictive terms “consisting essentially of” and “consisting of.” With the term “consisting essentially of” it is understood that the epitope/antigen of the present invention “substantially” comprises the indicated sequence as “essential” element. Additional sequences may be included at the 5′ end and/or at the 3′ end. Accordingly, a polypeptide “consisting essentially of” sequence X will be novel in view of a known polypeptide accidentally comprising the sequence X. With the term “consisting of” it is understood that the polypeptide, polynucleotide and/or antigen according to the invention corresponds to at least one of the indicated sequence (for example a specific sequence indicated with a SEQ ID Number or a homologous sequence or fragment thereof).

A person skilled in the art will appreciate that the present invention may be practiced without undue experimentation according to the method given herein. The methods, techniques and chemicals are as described in the references given or from protocols in standard biotechnology and molecular biology text books.

According to a preferred aspect, the present invention provides isolated chimeric monoclonal antibodies and related binding proteins that bind specifically to influenza A subtype H5N1. The antibodies according to any aspect of the present invention may be monoclonal antibodies (Mab) which may be a substantially homogeneous population of antibodies derivable from a single antibody-producing cell. Thus, all antibodies in the population may be identical and may have the same specificity for a given epitope. The specificity of the Mab responses provides a basis for an effective treatment against influenza A subtype H5N1 infection and/or at least one influenza A subtype H5N1-linked disease. Monoclonal antibodies and binding proteins derived therefrom also have utility as therapeutic agents.

The antibodies according to any aspect of the present application provide at least one anti-influenza A subtype H5N1 antibody which is capable of neutralizing influenza A subtype H5N1 infection and inhibiting cell-to-cell spread. These antibodies according to any aspect of the present application may be used as prophylactic and/or therapeutic agent(s) for the treatment of influenza A subtype H5 and influenza A subtype H5N1-linked diseases.

In a first aspect, there is provided an isolated chimeric antibody, variant, mutant or fragment thereof, wherein the antibody, variant, mutant or fragment thereof is capable of specifically binding to a conformational (non-linear) epitope on influenza A virus subtype H5N1, wherein the conformational epitope comprises an antigenic site comprising, consisting essentially of or consisting of the amino acid sequence I/LVKK.

Antibodies raised to this region of the influenza A virus according to the invention have been found to inhibit virus infectivity. The antibodies of the invention bind to the HA1 globular head and appear to inhibit the fusion process during virus uncoating.

In a preferred embodiment of the disclosure, the conformational epitope comprises three antigenic sites, wherein a first site comprises, consists essentially of or consists of the amino acid sequence I/LVKK, a second site comprises, consists essentially of or consists of the amino acid sequence WLL and the third site comprises, consists essentially of or consists of the amino acid sequence EWSYIV. These residues map to the membrane distal vestigial esterase subdomain of HA1.

More specifically, the conformational epitope binding site sequences consist of I/LVKK (SEQ ID NO: 17/18), WLL (SEQ ID NO: 19) and EWSYIV (SEQ ID NO: 20).

It is important in the clinical setting that the antibody does not itself elicit an immune response in the subject. Therefore, it has become common practice to minimise or eliminate the immunogenicity of antibodies raised in other species used for human treatment by humanizing them.

Techniques have been developed for the production of humanized antibodies [See Examples section herein]. An immunoglobulin light or heavy chain variable region consists of a “framework” region interrupted by three hyper variable regions, referred to as complementarity determining regions (CDRs). The extent of the framework region and CDRs have been precisely defined [see, “Sequences of Proteins of Immunological Interest”, Kabat, E. et al., U.S. Department of Health and Human Services (1983), incorporated herein by reference in their entirety]. Briefly, humanized antibodies are antibody molecules from non-human species having one or more CDRs from the non-human species and a framework region from a human immunoglobulin molecule.

Another preferred embodiment of the disclosure relates to the antibody or fragment thereof being a humanized antibody. More preferably, the antibody, variant, mutant or fragment thereof is a mouse-human chimeric antibody.

Antibody fragments that contain the idiotype of the antibody molecule can be generated by known techniques. For example, such can be produced by pepsin digestion of the antibody molecule; the Fab fragments can be generated by reducing the disulfide bridges of the F(ab)2 fragment, and the Fab fragments which can be generated by treating the antibody molecule with papain and a reducing agent. Such antibody fragments can be generated from any of the antibodies of the invention.

Chimeric antibodies can be produced by splicing the genes from a mouse antibody molecule of appropriate antigen specificity together with genes from a human antibody molecule of appropriate biological activity [Hanson et al., 2006, incorporated herein by reference, and Examples section herein]. For example, the genes from a mouse antibody molecule specific for a influenza A HA epitope can be spliced together with genes from a human antibody molecule of appropriate biological activity. A chimeric antibody is a molecule in which different portions are derived from different animal species, such as those having a variable region derived from a murine mAb and a human immunoglobulin constant region [Cabilly et al., U.S. Pat. No. 4,816,567; and Boss et al., U.S. Pat. No. 4,816,397, incorporated herein by reference]. Chimeric antibodies are also those that contain a human Fc portion and a murine (or other non-human) Fv portion.

In another preferred embodiment, the chimeric antibody comprises at least one variable heavy chain and at least one variable light chain, wherein the heavy chain comprises the VH domain of mouse monoclonal antibody 9F4, a variant, mutant or fragment thereof and the light chain comprises the VL domain of mouse monoclonal antibody 9F4, a variant, mutant or fragment thereof.

Preferably, the chimeric antibody comprises the mouse VH domain ligated to human IgG₁ heavy chain constant (CH) domain and the mouse VL domain ligated to human light chain kappa constant (CL) domain; or wherein the antibody comprises the mouse VH domain ligated to human IgA₁ heavy chain constant (CH) domain and the mouse VL domain ligated to human light chain kappa constant (CL) domain.

There are four different IgG subclasses (IgG1, 2, 3, and 4) in human. Although there is about 95% similarity in the sequence of their heavy chain constant (CH) domain, the structure of the hinge regions is relatively different, resulting in unique biological properties of each of the subclass. Since the antigen binding property of an antibody is usually independent of the heavy chain constant (CH) domain and light chain constant (CL) domain of the antibody, it is possible to change them to a matching pair without affecting antigen binding. We have shown herein that the heavy chain constant (CH) domain and light chain constant (CL) domain of the original mouse IgG_{2b} was successfully changed to human IgG₁ without any loss in antigen binding. Hence, it may be expected that replacing the original mouse IgG_{2b} of 9F4 with human IgG2, 3 or 4 will not have any significant impact on antigen binding. However, the effector functions and half-lives are expected to be different and the in vivo neutralization activities of each of these mouse-human chimeric antibodies have to be examined experimentally.

More preferably, the antibody comprises; (a) a variable heavy chain comprising the amino acid sequence of SEQ ID NO: 1, a variant, mutant or fragment thereof, and a variable light chain comprising the amino acid sequence of SEQ ID NO: 2, a variant, mutant or fragment thereof, for mouse-human chimeric IgG₁ antibody, or
(b) a variable heavy chain comprising the amino acid sequence of SEQ ID NO: 1, a variant, mutant or fragment thereof, and a variable light chain comprising the amino acid sequence of SEQ ID NO: 2, a variant, mutant or fragment thereof, for mouse-human chimeric IgA₁ antibody.

In a preferred embodiment the chimeric antibody comprises (a) a variable light chain comprising a sequence having at least 90% sequence identity to SEQ ID NO: 1 and a variable heavy chain comprising a sequence having at least 90% sequence identity to SEQ ID NO: 2, or

(b) a variable light chain comprising a sequence having at least 90% sequence identity to SEQ ID NO: 5 and a variable heavy chain comprising a sequence having at least 90% sequence identity to SEQ ID NO: 6.

Preferably, in (a) the heavy chain sequence of SEQ ID NO: 1 is encoded by a nucleic acid that has at least 90% sequence identity to the nucleotide sequence of SEQ ID NO: 3 and the light chain SEQ ID NO: 2 is encoded by a nucleic acid that has at least 90% sequence identity to the nucleotide sequence of SEQ ID NO: 4, and in (b) the heavy chain sequence of SEQ ID NO: 5 is encoded by a nucleic acid that has at least 90% sequence identity to the nucleotide sequence of SEQ ID NO: 7 and the light chain SEQ ID NO: 6 is encoded by a nucleic acid that has at least 90% sequence identity to the nucleotide sequence of SEQ ID NO: 8.

More preferably, in (a) the heavy chain sequence of SEQ ID NO: 1 is encoded by a nucleic acid having the nucleotide sequence of SEQ ID NO: 3 and the light chain SEQ ID NO: 2 is encoded by a nucleic acid having the nucleotide sequence of SEQ ID NO: 4, and in (b) the heavy chain sequence of SEQ ID NO: 5 is encoded by a nucleic acid having the nucleotide acid sequence of SEQ ID NO: 7 and the light chain SEQ ID NO: 6 is encoded by a nucleic acid having the nucleotide sequence of SEQ ID NO: 8.

Suitable oligonucleotide primers for amplifying and cloning the heavy and light chain variable domains of 9F4 into a human IgG1 or IgA1 cloning plasmids described herein are as follows:

1) Primers Used for Cloning of xi-IgA₁-9F4
A) Heavy chain: Forward primer SEQ ID NO: 9 and Reverse primer SEQ ID NO: 10.
B) Light chain: Forward primer SEQ ID NO: 11 and Reverse primer SEQ ID NO: 12.
   2) Primers Used for Cloning of xi-IgA₁-9F4
A) Heavy chain: Forward primer SEQ ID NO: 13 and Reverse primer SEQ ID NO: 14.
B) Light chain: Forward primer SEQ ID NO: 15 and Reverse primer SEQ ID NO: 16.

In another preferred embodiment, the chimeric antibody has the H5N1 binding and neutralization characteristics of mouse monoclonal antibody 9F4, xi-IgG₁-9F4 or xi-IGA₁-9F4. Preferably the antibody of the invention has the H5 binding and neutralization characteristics of chimeric monoclonal antibody xi-IgG₁-9F4 or xi-IGA₁-9F4, which have the ability to neutralize pseudovirus particles bearing H5 from clades 1, 2.1, 2.2 and 2.3.4.

In another preferred embodiment, the antibody binds to clade 2.3.4 H5N1 HA.

In another preferred embodiment, the chimeric antibody is linked with at least one drug, preferably an anti-viral drug. For example, the anti-viral drug may be a neuraminidase inhibitor. More particularly, the neuraminidase inhibitor may be Oseltamivir or Zanamivir.

In another aspect of the disclosure, there is provided a method of producing at least one mouse-human chimeric antibody which binds influenza A virus subtype H5N1, the method comprising the steps of:
a. Ligating a 9F4 VH domain nucleic acid encoding SEQ ID NO: 1 to a human IgG₁ CH domain and ligating a 9F4 VL domain nucleic acid encoding SEQ ID NO: 2 to a human CL domain in a single IgG₁ constant region expression vector; or
b. Ligating a 9F4 VH domain nucleic acid encoding SEQ ID NO: 5 to human IgA₁ CH domain in a first cloning vector, and ligating a 9F4 VL domain nucleic acid encoding SEQ ID NO: 6 to a human CL domain in a second cloning vector;
c. Transfecting the resulting chimeric construct or constructs into a suitable cell line; and
d. Collecting cell culture supernatants and extracting and purifying the chimeric antibody.

In a preferred embodiment, there is provided an isolated nucleic acid molecule comprising the variable heavy chain nucleotide sequence of SEQ ID NO: 3 and the variable light chain nucleotide sequence of SEQ ID NO: 4. More preferably the isolated nucleic acid molecule is a chimeric antibody construct. More preferably, the chimeric antibody construct is an IgA₁ CH domain cloning vector.

In a preferred embodiment, there is provided an isolated nucleic acid molecule comprising the heavy chain nucleotide sequence of SEQ ID NO: 7 and the light chain nucleotide sequence of SEQ ID NO: 8. More preferably the isolated nucleic acid molecule is a chimeric antibody construct. More preferably, the chimeric antibody construct is an IgG₁ CH domain cloning vector.

According to a further preferred aspect of the invention, there is provided at least one conformational epitope of influenza A subtype H5N1, wherein the conformational epitope is capable of being recognized by at least one antibody according to any aspect of the present invention.

In another aspect of the disclosure, there is provided an isolated chimeric antibody produced according to the method described herein.

In another aspect of the disclosure, there is provided the herein described antibody or a fragment thereof for use in medicine.

In another aspect of the disclosure, there is provided a method of treatment of influenza A subtype H5N1 disease, the method comprising administering to a subject in need thereof an efficacious amount of at least one chimeric antibody, variant, mutant or a fragment thereof according to the invention.

In a preferred embodiment of the method of treatment the at least one antibody or a fragment thereof is administered in combination with one or more other antibodies directed to Influenza A which bind to virus epitopes that do not compete with binding of same. In this respect, the use of two or more antibodies which do not compete for the same influenza A subtype H5N1 epitope should be more therapeutically effective and reduce the likelihood of escape mutants.

In another aspect of the disclosure there is provided the use of the antibody or a fragment thereof according to the invention for the preparation of a medicament for the treatment of influenza A subtype H5N1 disease. Preferably the medicament comprises a chimeric antibody comprising (a) a variable heavy chain comprising
the amino acid sequence of SEQ ID NO: 1, or fragment thereof, and a variable light chain comprising the amino acid sequence of SEQ ID NO: 2, or fragment thereof, for mouse-human chimeric IgG₁ antibody, or
(b) a variable heavy chain comprising the amino acid sequence of SEQ ID NO: 1, or fragment thereof, and a variable light chain comprising the amino acid sequence of SEQ ID NO: 2, or fragment thereof, for mouse-human chimeric IgA₁ antibody.

In another aspect of the disclosure there is provided a kit for treating influenza A subtype H5N1 disease, the kit comprising at least one antibody, variant, mutant or a fragment thereof according to any aspect of the invention.

In another preferred embodiment there is provided an isolated nucleic acid molecule encoding:
(a) at least one variable heavy chain of the antibody or a fragment thereof, wherein the heavy chain comprises the amino acid sequence of SEQ ID NO: 1, a variant, mutant or fragment thereof; and at least one variable light chain of the antibody, variant, mutant or a fragment thereof, wherein the light chain comprises the amino acid sequence of SEQ ID NO: 2, a variant, mutant or fragment thereof, or
(b) at least one variable heavy chain of the antibody, variant, mutant or a fragment thereof, wherein the heavy chain comprises the amino acid sequence of SEQ ID NO:

5, a variant, mutant or fragment thereof; and at least one variable light chain of the antibody, variant, mutant or a fragment thereof, wherein the light chain comprises the amino acid sequence of SEQ ID NO: 6, a variant, mutant or fragment thereof, and

wherein the nucleic acid molecule encodes (a) an IgG₁ chimeric antibody or (b) an IgA₁ chimeric antibody, respectively.

Preferably, in regard to the isolated nucleic acid molecule, in a) the heavy chain nucleic acid sequence has at least 80%, at least 90%, or at least 95% sequence identity to SEQ ID NO: 3 and the light chain nucleic acid sequence has at least 80%, at least 90%, or at least 95% sequence identity to SEQ ID NO: 4 as listed in Table 1; and in b) the heavy chain nucleic acid sequence has at least 80%, at least 90%, or at least 95% sequence identity to SEQ ID NO: 7 and the light chain nucleic acid sequence has at least 80%, at least 90%, or at least 95% sequence identity to SEQ ID NO: 8, as listed in Table 2.

Preferably, in regard to the isolated nucleic acid molecule, in a) the heavy chain nucleic acid sequence has at least 90% sequence identity to SEQ ID NO: 3 and the light chain nucleic acid sequence has at least 90% sequence identity to SEQ ID NO: 4; and in b) the heavy chain nucleic acid sequence has at least 90% sequence identity to SEQ ID NO: 7 and the light chain nucleic acid sequence has at least 90% sequence identity to SEQ ID NO: 8.

More preferably, in regard to the isolated nucleic acid molecule, in a) the heavy chain nucleic acid sequence is SEQ ID NO: 3 and the light chain nucleic acid sequence is SEQ ID NO: 4; and in b) the heavy chain nucleic acid sequence is SEQ ID NO: 7 and the light chain nucleic acid sequence is SEQ ID NO: 8.

In another aspect of the invention, there is provided an expression vector comprising the chimeric nucleic acid molecule according to any aspect of the invention. For example, suitable expression vectors are disclosed herein in the Examples.

In another aspect of the invention, there is provided a host cell comprising the expression vector according to any aspect of the invention.

### EXAMPLES

Standard molecular biology techniques known in the art and not specifically described were generally followed as described in Sambrook and Russell, Molecular Cloning: A Laboratory Manual, Cold Springs Harbor Laboratory, New York (2001).

### Materials and Methods

### Cell Lines and Transient Transfection

293FT cells were from Invitrogen. MDCK and HeLa cells were from American Type Cell Collection (Manassas, Va., USA). All cell lines were cultured at 37° C. in 5% CO₂ in Dulbecco's modified Eagle's medium (DMEM) supplemented with 10% fetal bovine serum. Growth media for 293FT and HeLa cells were further supplemented with non-essential amino acids and antibiotics.

Transient transfection experiments were performed using Lipofectamine™ 2000 reagent (Invitrogen), according to manufacturer's instruction. Where needed, transfected cells were used directly for immunofluorescence experiments or lysed with a lysis buffer containing 150 mM NaCl, 50 mM Tris (pH 7.5), 0.5% NP-40, 0.5% deoxycholic acid (sodium), 0.025% SDS, and 1 mM phenylmethylsulfonyl fluoride for downstream ELISA and Western blot analysis. For the endoglycosidase H (EndoH) treatment, the lysates from transfected cells were treated with EndoH enzyme (Roche Diagnostics) at 37° C. for 2 h before Western blot analysis. For the control, samples were treated in the same manner except no enzyme was added.

### HA Expressing Plasmids and HA Recombinant Proteins

The HA expressing plasmids used in this study contained full length HA coding sequences from Hatay04 [clade 1 virus: A/chicken/Hatay/2004(H5N1)], VN04 [clade 1 virus: A/Vietnam/1203/2004(H5N1)], Indo05 [clade 2.1 virus: A/Indonesia/5/2005(H5N1)], India06 [clade 2.2 virus: A/chicken/India/NIV33487/2006(H5N1)] and DLO6 [clade 2.3.4 virus: A/duck/Laos/3295/2006(H5N1)] (Genbank accession numbers AJ867074, EF541403, EU146622, EF362418 and DQ845348, respectively).

Purified HA1 recombinant protein of India06 was purchased from Sinobiologicals, China. Recombinant peptide ²⁵⁹KIVKKGDSTIM²⁶⁸ (based on H3 numbering) (SEQ ID NO: 21) was purchased from BioGenes, Berlin.

### Rabbit and Mouse Abs

Mouse MAb 9F4 and rabbit anti-H5N1 HA polyclonal antibodies (Rb anti-HA) were generated in previous studies (Oh et al., 2010; Shen et al., 2008). For all assays, mouse MAb 8F8, specific for M1 of Hatay04, was used as a negative control antibody and was generated using previously established protocol (Oh et al., 2010). Mouse MAb for β-actin was purchased from Sigma.

Cloning and Expression of xi-IgG₁-9F4 and xi-IgA₁-9F4

Total RNA was extracted from MAb 9F4 hybridoma by using RNeasy kit (Qiagen) and used for first strand cDNA synthesis using SuperScript II reverse transcriptase (Invitrogen). Variable heavy (VH) and variable light (VL) genes were amplified in subsequent PCR using Expand High Fidelity PCR (Roche). The Ig-primer set (Novagen) was used for these reactions, according to manufacturer's instruction. PCR products were cloned into pCRII-TOPO vector using the TOPO TA cloning kit (Invitrogen) and sequencing was performed using BigDye® Terminator v3.1 Cycle Sequencing Kit (Applied Biosystems). Variable regions were then defined using the IMGT database (Ehrenmann et al., 2010).

The amino acid and nucleotide regions of 9F4 used to produce the chimeric antibodies xi-IgG₁-9F4 and xi-IgA1-9F4 are shown in Tables 1 and 2.

TABLE 1
amino acid and nucleotide sequences used to
produce the chimeric antibody xi-IgG₁-9F4

| Name of gene | | |
|---|---|---|
| product isolated | | |
| from 9F4 | | 9F4 specific protein |
| hybridoma cell line | DNA sequence of PCR product obtained¹ | sequence in xi-9F4 |
| Variable region of | ATGGAATGGACCTGGGTTATCCTCTTCCTGTTGTCA | MEWTWVILFLLSVTAGVHSQ |
| immunoglobulin | GTAACTGCAGGTGTCCACTCCCAGGTCCAGCTGCAG | VQLQQSEAELARPGASVKMS |
| heavy chain (VH) | CAGTCTGAAGCTGAACTGGCAAGACCTGGGGCCTCA | CKASGFTLTTFTIHWVKQRP |
| | GTGAAGATGTCCTGCAAGGCTTCTGGCTTCACCTTG | GQGLEWIGYINPRSGYTDYN |
| | ACTACCTTCACGATCCACTGGGTAAAACAGAGGCCT | QKFKDNTTLTVDKSSSTAYM |
| | GGACAGGGTCTGGAATGGATTGGATACATTAATCCT | QLSSLTSEDSAVFYCARSYY |
| | CGCAGTGGATATACTGACTACAATCAGAAGTTCAAG | DYDVFDYWGQGTTLTVSSAK |
| | GACAATACCACATTGACTGTAGACAAATCCTCCAGC | TTPPPVYPLAPGSLGR |
| | ACAGCCTACATGCAACTGAGCAGCCTGACATCTGAG | (SEQ ID NO: 1) |
| | GACTCTGCGGTCTTTTACTGTGCAAGATCCTACTAT | underlined) |
| | GATTACGACGTCTTTGACTACTGGGGCCAAGGCACC | |
| | ACTCTCACAGTCTCCTCAGCCAAAACAACACCCCCA | |
| | CCCGTCTATCCATTGGCCCCTGGAAGCTTGGGAAGG | |
| | GC | |
| | (SEQ ID NO: 3 underlined) | |
| Variable region of | ATGAGGCCTTCGATTCAGTTCCTGGGGCTCTTGTTG | MRPSIQFLGLLLFWLHASQC |
| immunoglobulin | TTCTGGCTTCATGCTTCTCAGTGTGACGTCCAGATG | DVQMTQSPSSLSASLGGKVT |
| light chain (VL) | ACACAGTCTCCATCCTCACTGTCTGCATCTCTGGGA | ITCTARQDINKYIAWYQHKP |
| | GGCAAAGTCACCATCACTTGCACGGCAAGGCAAGAC | GKGPRLLIHYTSTLQPGIPS |
| | ATTAACAAGTATATCGCTTGGTACCAACACAAGCCT | RFSGSGSGTDYSFTISNLEP |
| | GGAAAAGGTCCTAGGCTGCTCATACATTACACATCT | EDIATYYCLQYDNLVTFGGG |
| | ACATTGCAGCCAGGCATCCCATCAAGGTTCAGTGGA | TKLELKRADAAPTVSIFPPS |
| | AGTGGGTCTGGGACAGATTATTCTTTCACCATCAGC | SKLGKGEF |
| | AACCTGGAGCCTGAAGATATTGCAACTTATTATTGT | (SEQ ID NO: 2 |
| | CTACAGTATGATAATCTGGTCACGTTCGGTGGTGGG | underlined) |
| | ACCAAACTGGAGCTGAAACGGGCTGATGCTGCACCA | |
| | ACTGTATCCATCTTCCCACCATCCAGTAAGCTTGGG | |
| | AAGGGCGAATTC | |
| | (SEQ ID NO: 4 underlined) | |

¹Underlined DNA fragment was cloned into vector and then used to produce xi-IgG₁-9F4.

TABLE 2
amino acid and nucleotide sequences used to produce the
chimeric antibody xi-IgA1-9F4

| Name of gene | | |
|---|---|---|
| product isolated | | |
| from 9F4 | | 9F4 specific protein |
| hybridoma cell line | DNA sequence of PCR product obtained¹ | sequence in xi-9F4 |
| Variable region of | ATGGAATGGACCTGGGTTATCCTCTTCCTGTTGTCA | MEWTWVILFLLSVTAGVHSQ |
| immunoglobulin | GTAACTGCAGGTGTCCACTCCCAGGTCCAGCTGCAG | VQLQQSEAELARPGASVKMS |
| heavy chain (VH) | CAGTCTGAAGCTGAACTGGCAAGACCTGGGGCCTCA | CKASGFTLTTFTIHWVKQRP |
| | GTGAAGATGTCCTGCAAGGCTTCTGGCTTCACCTTG | GQGLEWIGYINPRSGYTDYN |
| | ACTACCTTCACGATCCACTGGGTAAAACAGAGGCCT | QKFKDNTTLTVDKSSSTAYM |
| | GGACAGGGTCTGGAATGGATTGGATACATTAATCCT | QLSSLTSEDSAVFYCARSYY |
| | CGCAGTGGATATACTGACTACAATCAGAAGTTCAAG | DYDVFDYWGQGTTLTVSSAK |
| | GACAATACCACATTGACTGTAGACAAATCCTCCAGC | TTPPPVYPLAPGSLGR |
| | ACAGCCTACATGCAACTGAGCAGCCTGACATCTGAG | (SEQ ID NO: 5 |
| | GACTCTGCGGTCTTTTACTGTGCAAGATCCTACTAT | underlined) |
| | GATTACGACGTCTTTGACTACTGGGGCCAAGGCACC | |
| | ACTCTCACAGTCTCCTCAGCCAAAACAACACCCCCA | |
| | CCCGTCTATCCATTGGCCCCTGGAAGCTTGGGAAGG | |
| | GC | |
| | (SEQ ID NO: 7 underlined) | |
| Variable region of | ATGAGGCCTTCGATTCAGTTCCTGGGGCTCTTGTTG | MRPSIQFLGLLLFWLHASQC |
| immunoglobulin | TTCTGGCTTCATGCTTCTCAGTGTGACGTCCAGATG | DVQMTQSPSSLSASLGGKVT |
| light chain (VL) | ACACAGTCTCCATCCTCACTGTCTGCATCTCTGGGA | ITCTARQDINKYIAWYQHKP |
| | GGCAAAGTCACCATCACTTGCACGGCAAGGCAAGAC | GKGPRLLIHYTSTLQPGIPS |
| | ATTAACAAGTATATCGCTTGGTACCAACACAAGCCT | RFSGSGSGTDYSFTISNLEP |
| | GGAAAAGGTCCTAGGCTGCTCATACATTACACATCT | EDIATYYCLQYDNLVTFGGG |
| | ACATTGCAGCCAGGCATCCCATCAAGGTTCAGTGGA | TKLELKRADAAPTVSIFPPS |
| | AGTGGGTCTGGGACAGATTATTCTTTCACCATCAGC | SKLGKGEF |
| | AACCTGGAGCCTGAAGATATTGCAACTTATTATTGT | (SEQ ID NO: 6 |
| | CTACAGTATGATAATCTGGTCACGTTCGGTGGTGGG | underlined) |
| | ACCAAACTGGAGCTGAAACGGGCTGATGCTGCACCA | |
| | ACTGTATCCATCTTCCCACCATCCAGTAAGCTTGGG | |
| | AAGGGCGAATTC | |
| | (SEQ ID NO: 8 underlined) | |

¹Underlined DNA fragment was cloned into vectors and then used to produce xi-IgA₁-9F4.

Variable region specific primers were designed to introduce Mfe1 and Xho1; and ApaL1 and Pst1 restriction sites to respectively flank MAb 9F4 VH and VL coding sequences by PCR. This enabled the ligation of MAb 9F4 VH to human IgG₁ heavy chain constant (CH) domain and MAb 9F4 VL to light chain kappa constant domain (CL) in a single IgG₁ constant region expression vector, as previously described (Hanson et al., 2006; incorporated herein by reference).

Variable region specific primers were designed to introduce EcoRI and NheI; and EcoRI and BsiWI restriction sites to respectively flank MAb 9F4 VH and VL coding sequences by PCR. This enabled the ligation of MAb 9F4 VH to the human IgA₁ CH domain within pFUSEss-CHIg-hA1 cloning plasmid and the MAb 9F4 VL to the human CL kappa domain within pFUSE2ss-CLIg-hK cloning plasmid. Both pFUSEss-CHIg-hA1 and pFUSE2ss-CLIg-hK cloning plasmids were purchased from InvivoGen.

1) Primers Used for Cloning of xi-IgA₁-9F4

A) Heavy chain:
9F4-H-Fwd:
(SEQ ID NO: 9)
5′-TAGCCCAGGTGCAATTGCAGCAGTCTGAAGCTGAA-3′
9F4-H-Rev:
(SEQ ID NO: 10)
5′-CCGCCCTCTCGAGACTGTGACAGTGGTGCCTTG-3′
B) Light chain:
9F4-L-fwd:
(SEQ ID NO: 11)
5′-GATCGAAGTGCACTCCGACGTCCAGATGACACAG-3′
9F4-L-rev:
(SEQ ID NO: 12)
5′-CCGTTTGATCTGCAGTTTGGTCCCACCACCGAA-3′

2) Primers Used for Cloning of xi-IgA₁-9F4

A) Heavy chain:
EcoRI-9F4-H-F(new):
(SEQ ID NO: 13)
5′-cggaattcgCAGGTCCAGCTGCA-3′
9F4H-NheI-R2:
(SEQ ID NO: 14)
5′-ctagctagcTGAGGAGACTGTGAGA
B) Light chain:
EcoRI-9F4L-F(new):
(SEQ ID NO: 15)
5′-cggaattcaGACGTCCAGATGACACAG-3′
9F4L-BsiW1-R2:
(SEQ ID NO: 16)
5′-gaacgtacgCCGTTTCAGCTCCAGT-3′

The actual fragment cloned into the vector for expression depends on the nature of the vector. In the present example, there was no need to start from the start codon ATG (Met) because there is a signal peptide in the vectors used. However, if another vector is used it may be necessary to start from the start codon.

After successful incorporation of MAb 9F4 sequences the chimeric constructs were transiently transfected into 293FT cells as described in the Lipofectamine™ 2000 (Invitrogen) reagent instructions. Expression of xi-IgG₁-9F4 was checked by immunofluorescence analysis while expression of xi-IgA₁-9F4 was checked by Western blot. Cell culture supernatants containing the respective chimeric MAb were collected at 24 h and 72 h post transfection. xi-IgG₁-9F4 and xi-IgA₁-9F4 MAbs were extracted from the pooled supernatants using a HiTrap™ protein G and HiTrap™ protein A columns (GE Healthcare) respectively, according to manufacturer's instructions. Purity of chimeric MAb was confirmed using SDS-PAGE analyses.

### Immunofluorescence Analysis

293FT or MDCK cells were seeded on coverslips 24 h prior to transient transfection with appropriate expression vectors. 24 h post transfection, the coverslips were washed twice with 1×PBS and cells were fixed with 4% paraformaldehyde (PFA) for 10 min. The coverslips were washed and cells were permeabilized with 0.1% Triton-X for 10 min, where necessary. The coverslips were washed and blocked with 1% BSA in 1×PBS for 30 min and incubated with primary MAbs diluted in 1% BSA in 1×PBS for 2 h. After washing to remove unbound MAbs, the cells were incubated with Alexa Fluor® 488-conjugated goat anti-human IgG or Alexa Fluor® 488 conjugated goat anti-mouse IgG (Molecular Probes®) for 1 h. Unbound secondary antibodies were removed by washing and the coverslips were mounted onto microscope slides using FluorSave™ mounting medium (Calbiochem, Merck Chemicals Ltd). Images were obtained using an epi-fluorescence microscope (Olympus BX60).

### Pseudotyped Lentiviral Particle Neutralization Assay

Lentiviral pseudotyped particles (HApp) harbouring the H5N1 HA glycoprotein were generated by co-transfection of 293FT cells with an H5N1 HA expression plasmid and the envelope-defective pNL4.3.Luc.R⁻ E⁻ lentiviral vector. HA sequences corresponding to the fore mentioned viruses were used to generate HApp as previously described (Oh et al., 2010). The neuraminidase gene from Hatay04 was also co-transfected to facilitate the release of pseudotyped particles from the 293FT cells. The culture supernatants were collected 24 h post transfection, and stored at −80° C. until use.

The pseudotyped particle neutralization assay was performed as previously described (Oh et al., 2010). Briefly, MAbs were serially diluted in DMEM and mixed with an equal volume of HApp for 1 h. The mixture was used to infect MDCK cells, which were seeded in 12-well plates 24 h prior to infection. The infected MDCK cells were incubated at 37° C. for 72 h and were lysed with 125 μl of 1× luciferase cell lysis buffer (Promega) per well. 50 μl of the lysate was tested for luciferase activity by the addition of 50 μl of luciferase substrate (Promega) and luminescence was measured with a luminometer (Infinite M200, Tecan). Viral entry, as reflected by the relative light units (RLU), was expressed as a percentage relative to the absence of antibody. Each experiment was performed in duplicate.

### ELISA

The total binding affinity of MAbs for specific test antigen was determined by direct ELISA. 96 well ELISA plates were coated with recombinant proteins or transfected cell lysates overnight at 4° C. and blocked with 5% milk for 1 h. Serially diluted MAbs in 2% milk were added to the plates and incubated for 1 h at 37° C. The plates were washed six times with phosphate-buffered saline (PBS) containing 0.05% Tween-20 (PBST) and incubated with horseradish-peroxidase-conjugated secondary antibodies (ThermoScientific) for 1 h at 37° C. The plates were washed six times with PBST before the reaction was visualized using the substrate 3,3′,5,5′-tetramethylbenzidine (TMB) (ThermoScientific) and stopped with 2 M H₂SO₄. The absorbance at 450 nm (A450) was measured using a plate reader.

### Statistics

Unpaired t-test was used to evaluate whether mouse and xi-mAbs differed in their binding or neutralizing activity from at least 3 sets of values for each ELISA and HApp neutralization assays.

### Syncytial Inhibition Assay

HeLa cells seeded on glass coverslips were transiently transfected with Hatay04-HA as described. The cells were then treated with two test concentrations of each MAb for 1 h at 37° C. in 5% CO₂, 48 h post transfection. Unbound MAbs were removed by washing the cells with 1×PBS prior to treatment with low pH buffer for 15 min at 37° C. in 5% CO₂. Excess low pH buffer was removed by washing and the cells were allowed to recover in growth media for 3 h at 37° C. in 5% CO₂. Cells were stained with CellMask Orange (Invitrogen) at 1:5000 dilution and fixed with 4% PFA. Finally, the cells were mounted onto glass slides using VectorShield mounting media with DAPI (Vector Laboratories). Images were obtained using an epi-fluorescence microscope (Olympus BX60).

### Epitope Mapping

N and C terminal deletion mutant constructs were generated by PCR within the Hatay04 construct. These mutants were transiently transfected into 293FT cells and expression levels were checked using Rb anti-HA. The ability of MAb 9F4 to bind to these mutants was evaluated by Western blot. In addition, MAb 9F4 binding to a combinatorial antigen library displayed on the surface of yeast was performed as previously described (Zuo et al., 2011) in order to determine the minimal HA binding fragment.

Initial data indicated the 9F4 epitope partly comprised the sequence ²⁵⁶I/LVKK²⁵⁹ (based on mature H5 numbering) and was likely a conformational epitope since linearization of H5 in western blot analysis results in the loss of binding. To guide experimental epitope mapping, two epitope prediction methods were used to identify potential antigenic fragments within VN04. The first method, BPAP, scores potential fragments based on hydrophillicity, accessibility and flexibility of amino acid residues. Additionally, fragments containing amino acids that are frequently found in experimentally validated linear epitopes (namely C, V and L) are given higher propensity scores (Kolaskar and Tongaonkar 1990). The second method, BEPro predicts discontinuous epitopes based half sphere exposure calculation, solvent accessibility and side chain orientation information from available three-dimensional structure of proteins and assigns a score to each residue (Sweredoski and Baldi 2008).

### Results

MAb 9F4 Binds and Prevents Viral Entry into MDCK Cells Mediated by HA of Clade 2.3.4 H5N1.

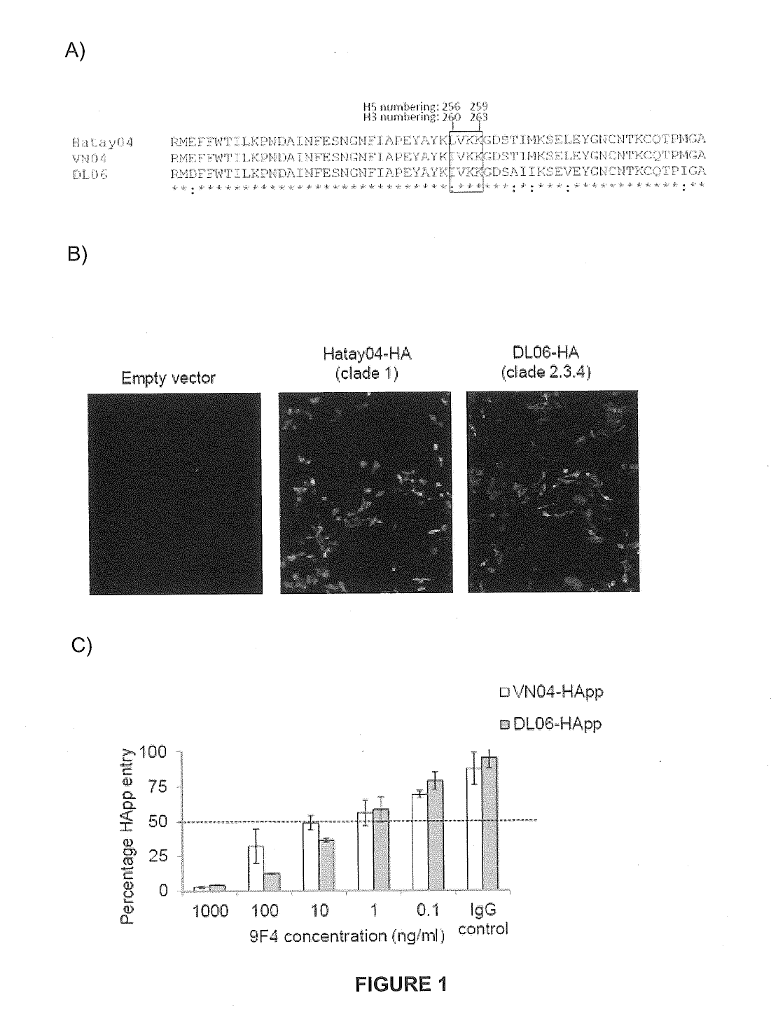

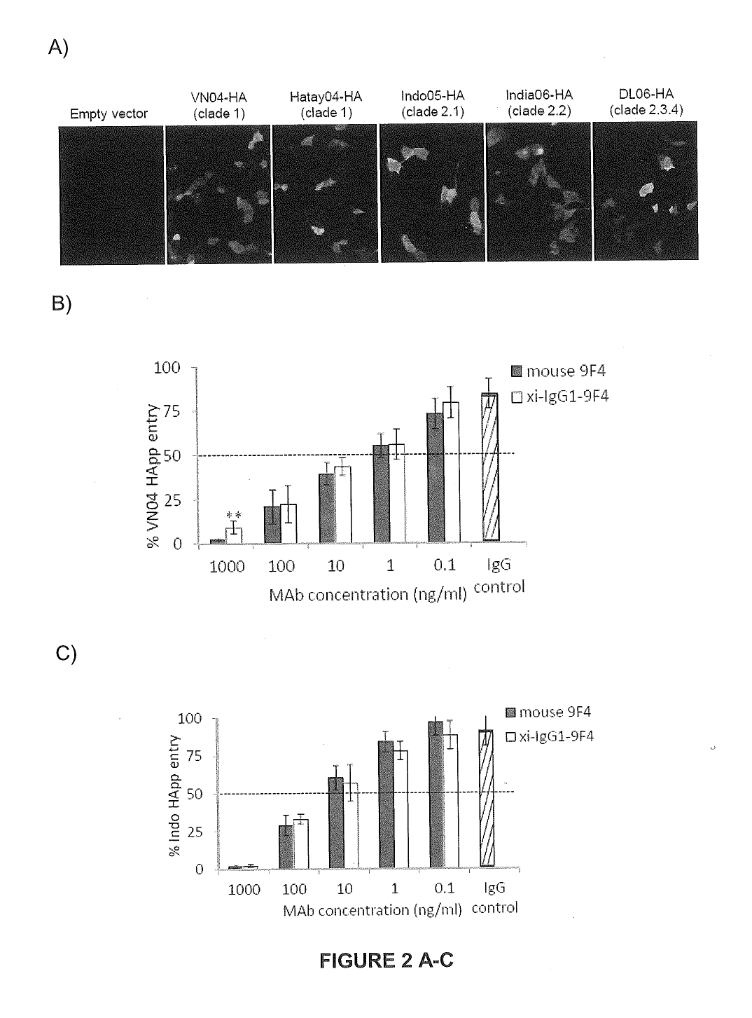

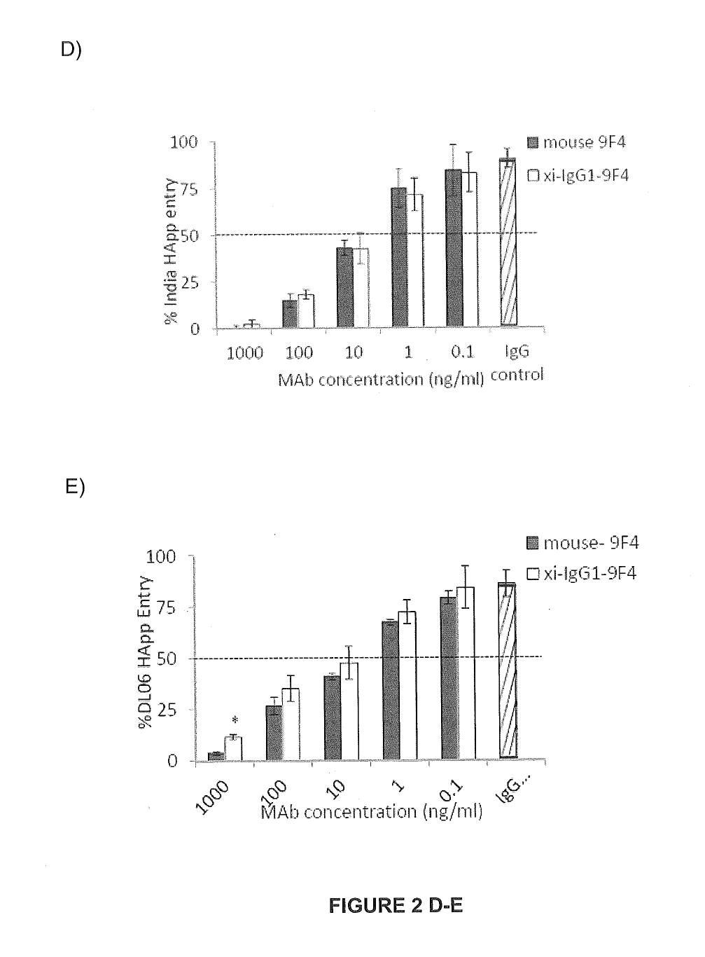

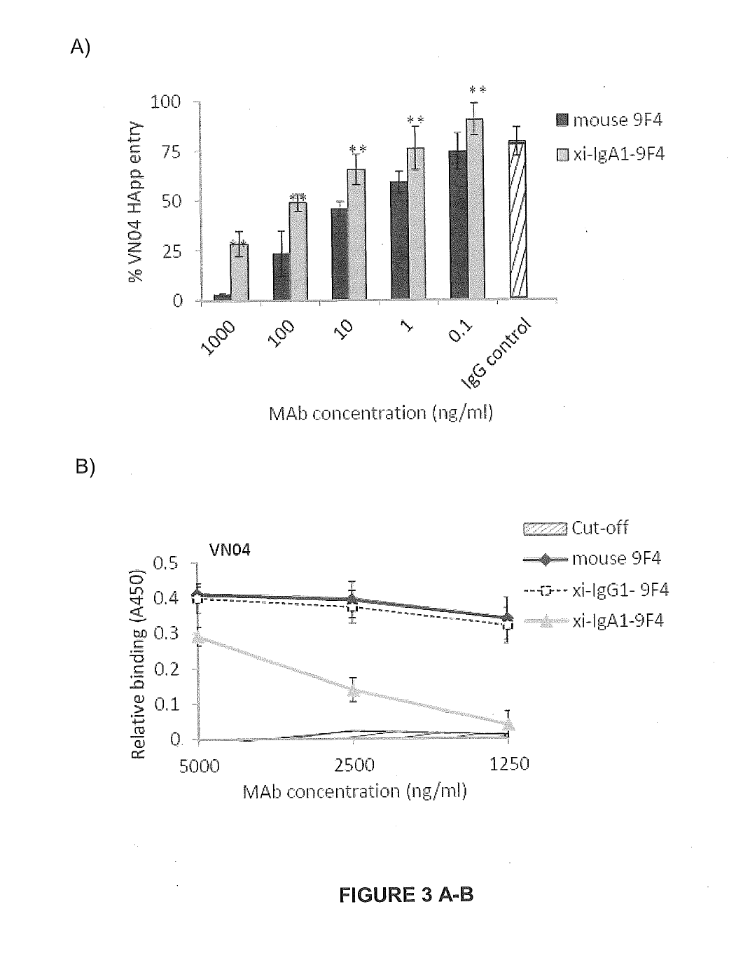

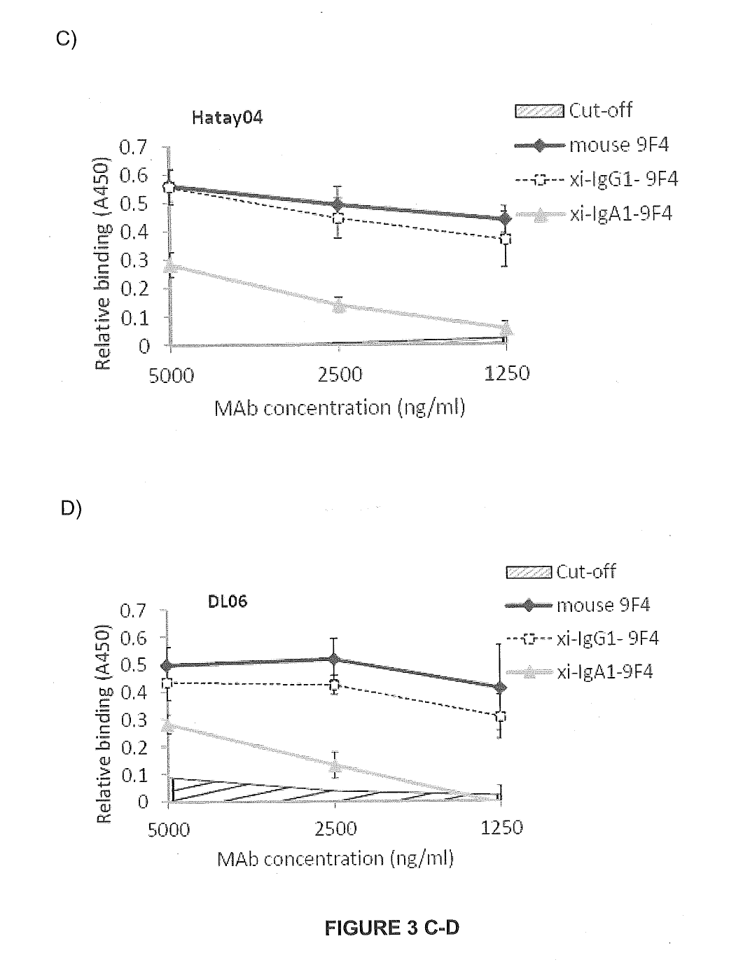

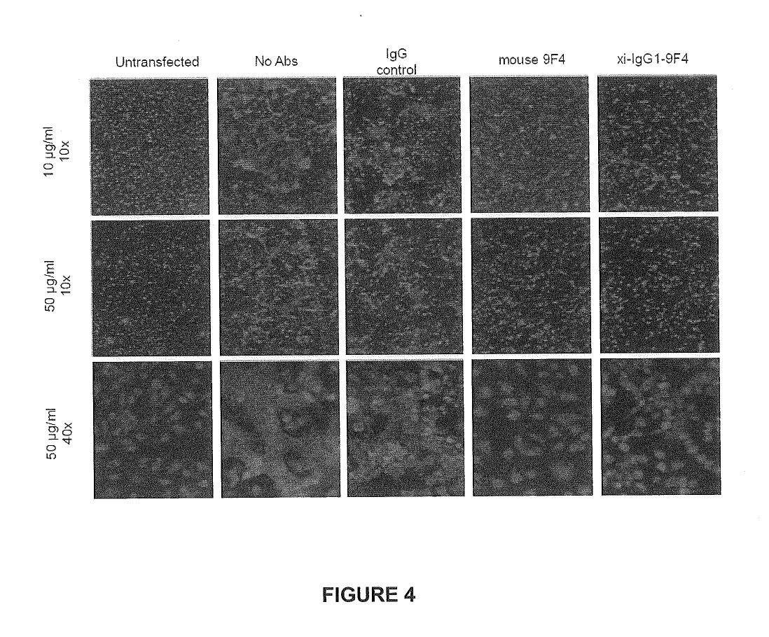

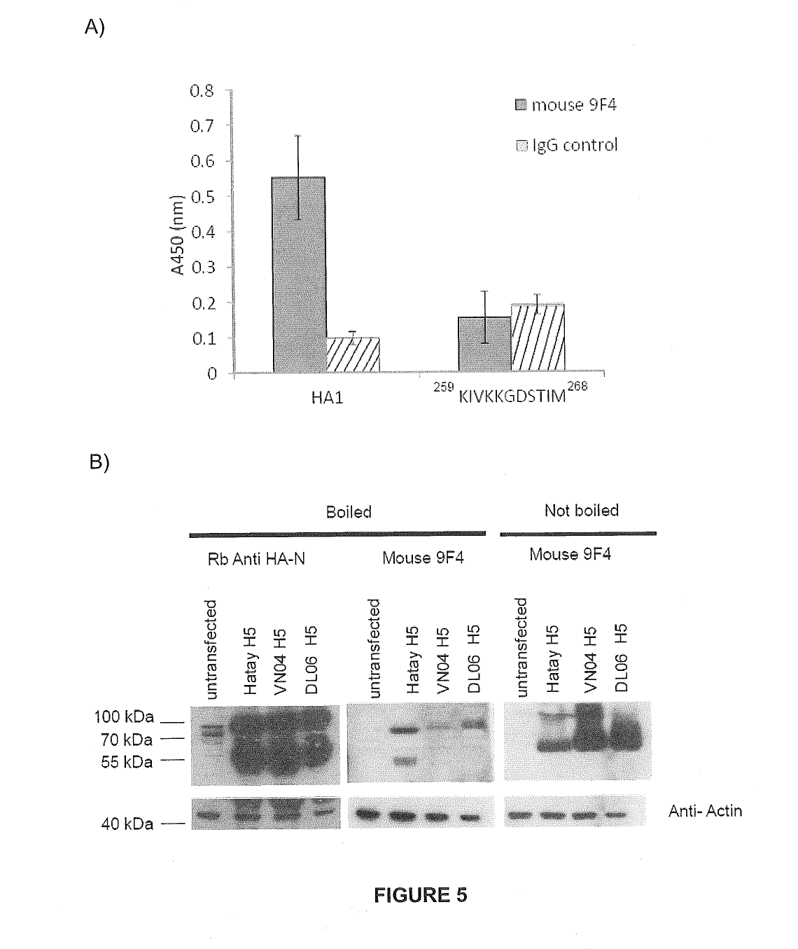

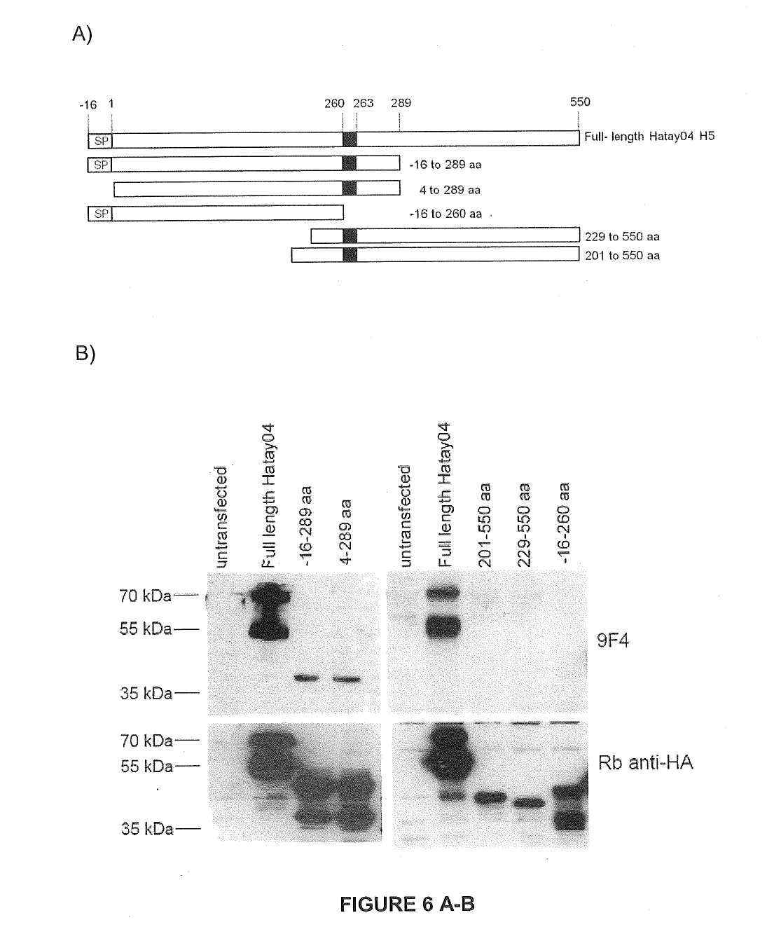

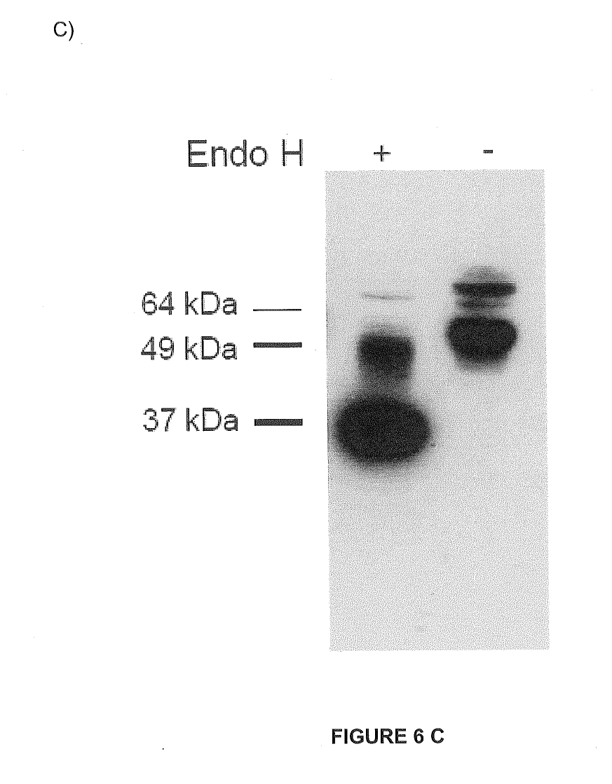

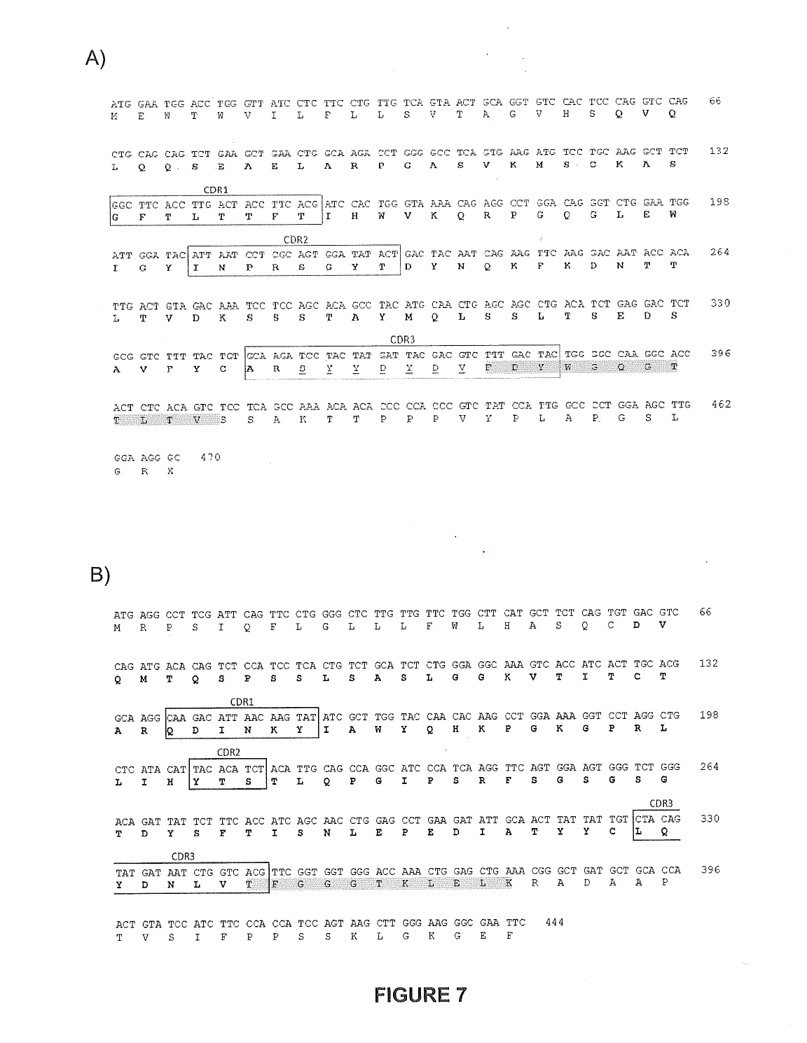

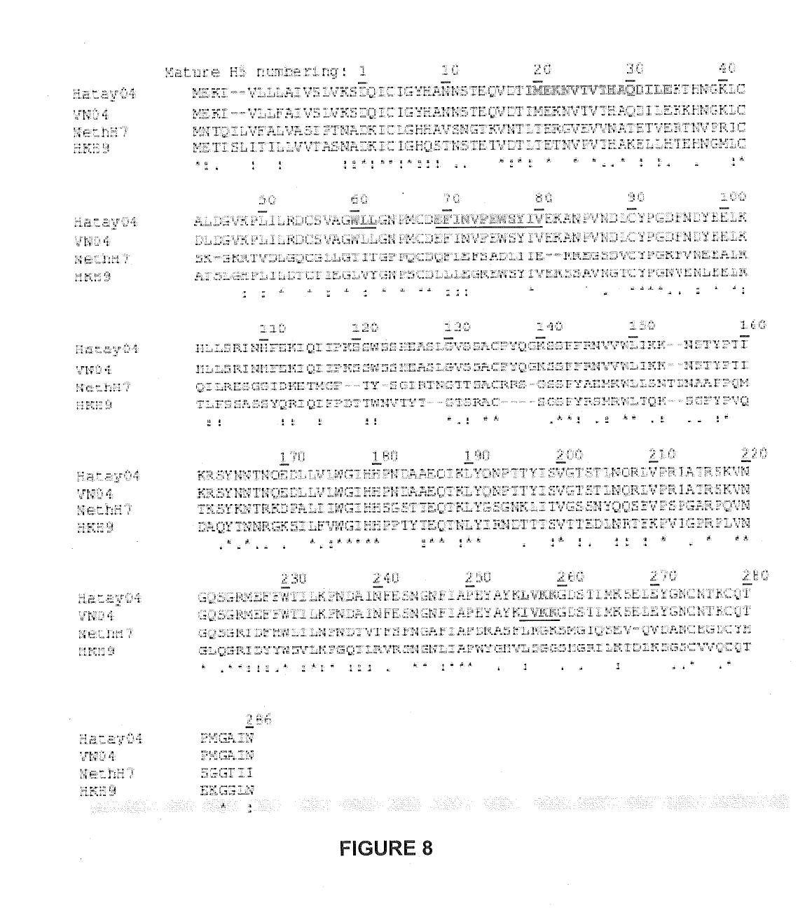

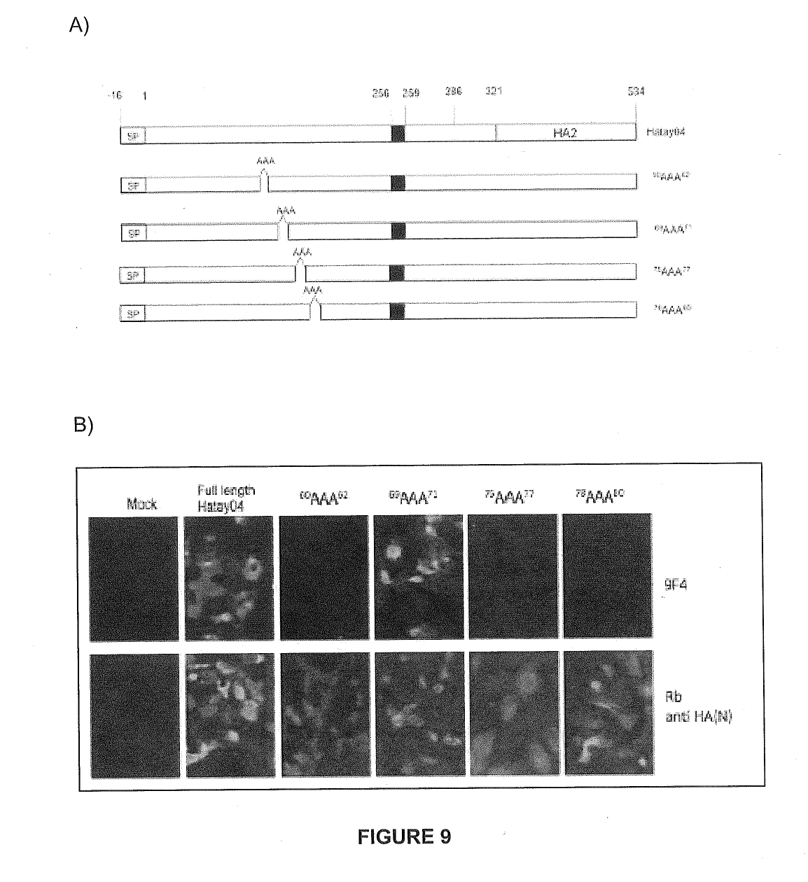

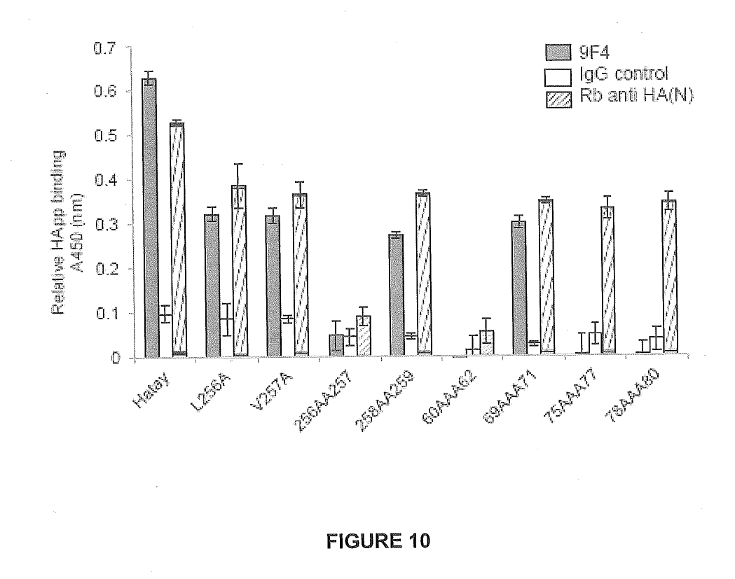

In 2007, a shift from clade 1 to clade 2.3.4 was reported for human H5N1 infections in Vietnam. Clade 2.3.4 viruses have since disseminated to Myanmar, Laos, China, Hong Kong and Bangladesh, where they have been isolated from humans and domestic birds. As clade 2.3.4 viruses retain the previously identified MAb 9F4 epitope site (FIG. 1A), we tested the ability of MAb 9F4 to bind to HA from a clade 2.3.4 H5N1 virus by immunofluorescence analysis on non-permeabilized cells. As shown in FIG. 1B, MAb 9F4 binds to native DL06-HA transiently expressed on the surface of MDCK cells.

The neutralizing ability of MAb 9F4 against HApp harboring DL06-HA was also examined. HApp contain the firefly luciferase reporter gene and permits the sensitive quantification of pseudovirus entry into host cells, which have been shown to display similar entry characteristics and neutralization titres as live virus (Garcia and Lai, 2011). MAb 9F4 inhibited the entry of DL06-HApp in a dose dependent manner, whereas the negative control antibody was unable to inhibit HApp entry into MDCK cells even when used at 10,000 ng/ml, which is 10 times higher than the highest concentration of MAb 9F4 used (FIG. 1C). The half-maximal inhibitory concentration (IC₅₀) for DL06-HApp was about 10 ng/ml, similar to clade 1 VN04-HApp as previously reported (Oh et al., 2010) and included in this experiment as a positive control.

Production of xi-IgG₁-9F4 and xi-IgA₁-9F4

The ability of MAb 9F4 to potently neutralize clade 1 and multiple clade 2 viruses from subclades 2.1, 2.2 (Oh et al., 2010) and 2.3.4 (FIG. 1C) makes it an attractive lead antibody for passive immunotherapy as viruses from these clades and subclades have caused human infection. To minimize potential rejection of MAb 9F4 for use in humans, a mouse-human chimeric form of MAb 9F4, named as xi-IgG₁-9F4, was generated. Firstly, the VH and VL chains of MAb 9F4 were obtained from the messenger RNA of the hybridoma by using PCR method (FIGS. 7 (A) and (B), respectively). To generate xi-IgG₁-9F4, specific gene fragments of VH and VL were then fused to the coding regions for CH chain of human IgG₁ and CL of the kappa chain respectively. The expression of xi-IgG₁-9F4 in 293FT cells was then checked by immunofluorescence staining. Positive immunofluorescence only in the presence of Alexa Fluor® 488-conjugated goat anti-human IgG confirmed the chimerization of MAb 9F4. No immunofluorescence was detected in the presence of Alexa Fluor® 488-conjugated goat anti-mouse IgG, indicating successful replacement of heavy and light chains to human forms (data not shown).

Similarly, a chimeric IgA₁ form of MAb 9F4 was generated by fusing 9F4 VH and VL to the coding regions for CH chain of human IgA₁ and CL of the kappa chain respectively. 293FT cells were used as producer cells and expression of xi-IgA₁-9F4 was detected using anti-human-IgA-HRP conjugate antibody in western blot analysis, indicating successful replacement of heavy and light chains to human forms (data not shown).

xi-IgG₁-9F4 Retains Binding and Neutralization Ability

Chimeric xi-IgG₁-9F4 antibody binding to native H5 HA from multiple H5N1 clades was detected by fluorophore-conjugated-anti-human IgG (FIG. 2A) but not in the presence of fluorophore-conjugated-anti-mouse IgG (data not shown). This indicates that conversion to xi-IgG₁ was successful and does not impede cross-clade binding.

Next, the pseudotyped lentivirus particle neutralization assay was used as a quantitative measure of xi-IgG₁-9F4 activity compared to mouse 9F4. As shown in FIG. 2B-E, both mouse and xi-IgG₁-9F4 inhibited the entry of HApp containing the HA of various H5N1 clades in a dose dependent manner. As expected, the negative control antibody was consistently unable to inhibit HApp entry even when used at 10,000 ng/ml. Neutralization of Indo05-HApp and India06-HApp mediated by mouse and xi-IgG₁-9F4 was similar at all MAb concentrations tested. Neutralization of VN04-HApp and DL06-HApp mediated by xi-IgG₁-9F4 differed from mouse 9F4 only at the highest concentration tested, where xi-IgG₁-9F4 reduces HApp entry by approximately 90% compared to complete neutralization by mouse 9F4. Nevertheless, xi-IgG₁-9F4 retains high neutralizing potency similar to mouse 9F4, with an approximate IC₅₀ of 10 ng/ml for all HApp tested.

Neutralization Ability of xi-IgA₁-9F4 is Decreased Due to Reduction in Binding Affinity

Unlike xi-IgG₁-9F4, the ability of chimeric xi-IgA₁-9F4 antibody to neutralize VN04-HApp was significantly reduced at all MAb concentrations tested. xi-IgA₁-9F4 only inhibited 75% of VN04-HApp entry at 1000 ng/ml and has an IC₅₀ of 100 ng/ml (FIG. 3A).

To account for the reduction in neutralization, we performed a comparative ELISA using total cell lysates from 293FT cells transiently expressing VN04-HA, Hatay04-HA and DL06-HA. These cell lysates contain all expressed forms of HA (precursor HA0 and mature disulfide-linked HA1-HA2 on cell surface) and were therefore suitable for assessing total binding affinity. The negative IgG control was used to determine the cut-off and the endpoint titre, defined as the MAb concentration that produces an A450 reading that is equivalent or lower than the cut-off, was determined (Frey et al., 1998).

While xi-IgG₁-9F4 and mouse 9F4 antibodies bound comparably to all H5 HA and at all MAb concentrations tested, binding by xi-IgA₁-9F4 was decreased (FIG. 3B-D). The endpoint titre for xi-IgA₁-9F4 was 1250 ng/ml for all H5 HA tested, whereas xi-IgG₁-9F4 and mouse 9F4 still exhibited strong binding at this concentration.

### Mouse and Mouse-Human Chimeric Form of MAb 9F4 Comparably Inhibit HA Mediated Fusion at Low pH.

It was previously suggested that MAb 9F4 inhibits fusion of viral and host endosomal membranes as MAb 9F4 did not show haemagglutination inhibition activity and was able to prevent low pH mediated HA conformational change (Oh et al., 2010). As xi-IgG₁-9F4 showed comparable binding and neutralizing activity as mouse-9F4, the ability of xi-IgG₁-9F4 to inhibit fusion was determined by means of a syncytial inhibition assay. Briefly, HeLa cells expressing HA were subjected to low pH treatment to allow HA-mediated cell membrane fusion. The resultant syncytia formation was analyzed by means of immunofluorescence staining. No syncytial formation was observed for untransfected cells (FIG. 4, first column), while large multinucleated syncytia bodies were observed for HA expressing HeLa cells in the absence of antibodies (FIG. 4 second column). It was observed that the pre-incubation of HA expressing HeLa cells with either mouse-9F4 and xi-IgG₁-9F4 reduced the amount and size of syncytia formation at a MAb concentration of 10 μg/ml and this reduction was more pronounced at 50 μg/ml (FIG. 4 fourth and fifth column). In contrast, the pre-incubation of HA expressing HeLa cells with an irrelevant mouse MAb 8F8 prior to low pH treatment did not prevent syncytia formation (FIG. 4 third column).

### MAb 9F4 Recognizes a Conformational Epitope

To allow easy comparison to other studies, the H3 numbering convention is used here. An epitope ²⁶⁰I/LVKK²⁶³ (based on H3 numbering) (SEQ ID NOs: 17 and 18) within the HA1 subunit is essential for the interaction with MAb 9F4 because full-length HA lacking this epitope could not bind MAb 9F4 (Oh et al., 2010). However, MAb 9F4 failed to react with linear peptide ²⁵⁹KIVKKGDSTIM²⁶⁸ (based on H3 numbering) (SEQ ID NO: 21) bearing ²⁶⁰I/LVKK²⁶³, although it reacted strongly with recombinant HA1 protein, which contains this peptide sequence, in ELISA analysis (FIG. 5A). This indicates that²⁶⁰I/LVKK²⁶³ is insufficient for binding. Hence, the ability of MAb 9F4 to bind to various transitional states of HA in western blot analysis was next examined. 293FT cells were transiently transfected with Hatay04-HA, VN04-HA and DL06-HA and the expression levels were verified using a rabbit polyclonal antibody raised against the N terminus of HA (FIG. 5B left panel). To compare the ability of MAb 9F4 to bind completely denatured and reduced HA versus partially denatured and reduced HA, western blot analysis was conducted under reducing conditions but with or without boiling. The results showed that MAb 9F4 bound to completely reduced and denatured HA (FIG. 5B middle panel) but this binding was very low compared to samples that were not boiled (FIG. 5B right panel), implying that MAb 9F4 has a binding preference towards native conformations of HA.

The contribution of residues upstream of ²⁶⁰I/LVKK²⁶³ to the interaction with MAb 9F4 epitope was next examined using truncated forms of Hatay04-HA proteins (FIG. 6A). As shown in FIG. 6B, MAb 9F4 bound to the N-terminal fragments of HA (−16 to 289aa and 4-289aa, based on H3 numbering) as well as the full-length HA protein (−16 to 550aa, based on H3 numbering). In contrast, no binding was observed for the N-terminal fragment of HA corresponding to −16 to 260aa (based on H3 numbering). This is consistent with our previous finding that residues 260 to 263 in HA are essential for MAb 9F4 interaction. However, it was observed that MAb 9F4 did not bind two C-terminal fragments of HA (201-550aa and 229-550aa, based on H3 numbering), although they both contain the ²⁶⁰I/LVKK²⁶³ epitope. Similar results were obtained using immunofluorescence analysis (data not shown). Next, MAb 9F4 was screened against a combinatorial HA antigen library displayed on the surface of yeast and the minimal binding fragment was found to span from 55-271aa (based on H3 numbering, data not shown). Collectively, the data suggests that MAb 9F4 binds to a conformation-dependent epitope on HA and additional residues upstream of ²⁶⁰I/LVKK²⁶³ are required for this interaction.

BPAP predicted a total of 15 fragments (Table 3) within the −16 to 286aa fragment (based on mature H5 numbering) (FIG. 8), which was previously found to be sufficient for 9F4 binding (Oh et al. 2010). Most VN04 residues predicted as likely epitopes were situated close to each other and can be clustered within 11 antigenic fragments. Both methods predicted at least part of the previously identified epitope ²⁵⁶I/LVKK²⁵⁹ (based on mature H5 numbering, shown underlined in Table 3).

TABLE 3
Antigenic fragments predicted using BPAP and BEPro.

| Residue | | HA |
|---|---|---|
| number | Sequence | domain |
| −12 to 6 | IVLLFAIVSLVKSDQICIG | SP/F |
| 19 to 34 | IMEKNVTVTHAQDILE | F |
| 38 to 62 | NGKLCDLDGVKPLILRDCSVAGWLL | VE |
| 69 to 82 | EFINVPEWSYIVEK | VE |
| 85 to 92 | PVNDLCYP | VE |
| 99 to 107 | EELKHLLSR | VE |
| 112 to 119 | EKIQIIPK | RBD |
| 125 to 140 | HEASLGVSSACPYQGK | RBD |
| 143 to 150 | FFRNVVWL | RBD |
| 170 to 177 | EDLLVLWG | RBD |
| 186 to 192 | EQTKLYQ | RBD |
| 196 to 202 | TYISVGT | RBD |
| 205 to 212 | LNQRLVPR | RBD |
| 248 to 258 | 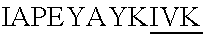 | RBD/VE |
| 274 to 280 | CNTKCQT | F |
| | | HA |
| Residue No | Sequence | domain |
| 1 to 15 | DQICIGYHANNSTEQ | F |
| 19 to 25 | IMEKNVT | F |
| 34 to 40 | EKTHNGK | F |
| 72 to 75 | INVP | F |
| 94 to 100 | NFNDYEE | VE |
| 103 to 110 | HLLSRINH | VE |
| 112 to 129 | EKIQIIPKSSWSSHEASL | RBD |
| 138 to 141 | QGKS | RBD |
| 151 to 171 | IKKNSTYPTIKRSYNNTNQED | RBD |
| 180 to 225 | HPNDAAEQIKLYQNPTTYISVGTSTL | RBD |
| 234 to 245 | KPNDAINFESNG | RBD |
| 255 to 261 | 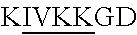 | RBD/VE |
| 268 to 275 | LEYGNCN | VE |

The previously identified epitope is shown underlined. SP = Signal peptide, F = fusion domain, VE = vestigial esterase domain, RBD = receptor binding domain. Domain assignment according to (Ha et al. 2002). Mature H5 numbering is employed in this table.

N-terminal truncated mutants were created to rule out the involvement of predicted N terminal antigenic sites. 9F4 bound to N- and C-terminal truncated mutants spanning 16-286aa and 4 to 286aa, which could also be detected by polyclonal Rb-anti HA(N) in immunofluorescence assay, suggesting that deletions did not affect proper folding of the mutant Hatay04 fragments. However, detection by Rb-anti HA(N) is abrogated in the 14-286aa mutant, indicating that large N-terminal deletions are deleterious. As a result, the involvement of 19-34aa was analysed using substitution or internal deletion mutations within the −16-286aa mutant (not shown). 9F4 retained binding to internal substitution and deletion mutants spanning 19-34aa (−16-286 I19A/M20A, −16-286Δ21-27 and −16-286Δ28-34) indicating that these residues are not involved in binding. These mutants were named based on mature H5 numbering.

Three criteria were then used to narrow down the epitopes to be tested. Firstly, since 9F4 is a homosubtypic MAb and does not bind H7 or H9 (data not shown), we reasoned that residues conserved between H5 HA but not in H7 or H9 are critical for 9F4 recognition. Secondly, critical residues should be in close proximity (within a 12 Å radius) to the ²⁵⁶I/LVKK²⁵⁹ in the 3D structure of H5. Thirdly, predicted fragments within the RBD were excluded since 9F4 does not inhibit hemagglutination (Oh et al. 2010). This process eliminated all but two predicted epitope sites ⁶⁰WLL⁶² and ⁶⁹EFINVPEWSYIV⁸⁰ (based on mature H5 numbering) (FIG. 8, underlined) within the vestigial esterase subdomain of HA1, which were selected for further testing.

Triple alanine (AAA) mutants (FIG. 9A) were constructed within full length Hatay04 HA to permit mutant HApp neutralization in future. The ability of 9F4 to bind these mutants was screened in immunofluorescence assay. As shown in FIG. 9B, positive immunofluorescence was only seen for Hatay04 and ⁶⁹AAA⁷¹ but not ⁶⁰AAA⁶², ⁷⁵AAA⁷⁷ and ⁷⁸AAA⁸⁰ (based on mature H5 numbering). All mutants could be detected by Rb anti HA(N), implying that the mutation did not affect overall protein fold and expression.

While attempting to create mutant HApp for the functional evaluation of 9F4 reactivity to these epitopes, it was discovered that the double alanine mutant ²⁵⁶AA²⁵⁷ [previously described in (Oh et al., 2010)] and ⁶⁰AAA⁶² could not be detected by Rb anti HA(N) in HApp ELISA analysis (FIG. 10) even though Rb anti HA(N) binding to ⁶⁰AAA⁶² was observed when over-expressed in MDCK cells (FIG. 9B) and previously described for ²⁵⁶AA²⁵⁷ (Oh et al. 2010). In contrast, ⁶⁹AAA⁷¹, ⁷⁵AAA⁷⁷ and ⁷⁸AAA⁸⁰ mutant HA could be detected in HApp, although binding is decreased compared to wild-type Hatay04 (p<0.05). Alanine mutants spanning the previously identified epitope: L256A, V257A and ²⁵⁸AA²⁵⁹ could be detected by Rb anti HA(N), albeit also at lower levels compared to wild-type Hatay04 (p<0.05). These findings imply that the ²⁵⁶LV²⁵⁷ motif as well as ⁶⁰WLL⁶² are required for HA incorporation into HApp. These mutants were named based on mature H5 numbering.

As shown in FIG. 10, the irrelevant IgG control did not react with either wild-type or mutant Hatay04. 9F4 binding to HApp mutants L256A, V257A, ²⁵⁸AA²⁵⁹ and ⁶⁹AAA⁷¹ was detectable in HApp ELISA but were lower than the positive control Rb anti HA(N). In contrast, 9F4 binding to wild-type Hatay04 HApp was higher than Rb anti HA(N), indicating that although mutations at these epitopes significantly reduced binding by 9F4, none of these epitopes alone completely abrogated HApp binding. In comparison, ²⁵⁶AA²⁵⁷, ⁷⁵AAA⁷⁷ and ⁷⁸AAA⁸⁰ completely demolished 9F4 binding, suggesting that these epitopes are important for 9F4 binding.

As HA glycosylation patterns change during antigenic evolution and can affect antibody binding, a further experiment was performed to determine if glycosylation of HA is essential for the interaction with MAb 9F4. The −16 to 289aa (based on H3 numbering) HA samples were treated with endoglycosidase H to remove N-linked hybrid or high mannose oligosaccharides prior to western blot analysis with MAb 9F4 and as shown in FIG. 6C, the removal of N-linked glycans did not reduce the binding of MAb 9F4.

### Discussion

Anti-H5N1 HA neutralizing antibodies can be classified according to their binding sites [reviewed in (Velkov et al., 2013)]. The majority of HA neutralizing MAbs targets the membrane distal receptor binding site (RBS) located on the globular head of HA1. Consequently, the selective antibody pressure drives antigenic drift and antibody escape. HA2 selective antibodies target the highly conserved fusion peptide region and therefore display broad cross-clade and varying degrees of heterosubtypic protection. However, a small number of neutralizing MAbs targeting non-RBS regions in HA1 have also been described. These MAbs are less well understood with some of them inhibiting the viral attachment step and others inhibiting post-attachment events. Some of these MAbs have been reported to provide homosubtypic cross-clade protection by binding conformation dependent epitopes (Cao et al., 2012; Hu et al., 2012). The novelty of these epitopes suggests that these MAb could be suitable in combination approaches with RBS selective or HA2 selective MAb in a polyclonal passive immunotherapeutic fashion and further discovery and evaluation of MAb within this obscure class is thus warranted.

MAb 9F4 is an example of neutralizing MAb targeting a non-RBS domain in HA1. MAb 9F4 protected mice against lethal H5N1 challenge and neutralizes clade 1, 2.1, 2.2 (Oh et al., 2010) and 2.3.4 HA-lentiviral pseudotyped particles (FIG. 1C). MAb 9F4 was found to be potently neutralizing, with an IC₅₀ of 10 ng/ml and IC₉₅ of 100 ng/ml, comparable to the anti-HA activity of other potently neutralizing MAbs (Cao et al., 2012; Corti et al., 2011; Du et al., 2013). To reduce immune rejection in humans, two chimeric forms of MAb 9F4 were created using recombinant molecular techniques. While xi-IgG₁-9F4 retained total binding affinity and neutralizing potency of mouse-9F4, xi-IgA₁-9F4 showed reduced binding and a 10-fold increase in the IC₅₀ value in the HApp neutralization assay. Since all three forms of the MAb 9F4 contain the same variable regions, the differences in binding affinity and neutralizing potency could be attributed to the differences in constant region domains. Although the variable antibody regions are usually expected to be sufficient for binding, constant regions have also been shown to participate through steric hindrances and inducing conformational changes in the targeted antigen (Nason et al., 2001).

As outcome of passive immunotherapy could be dependent on the efficacy by which passively transferred MAbs reach the sites of viral replication, 9F4 was reformatted into two chimeric isotypes in this study.

The degree of protection observed by parentally administered IgG MAbs in mice could be due in part to the disseminated nature of viral replication in murine models. Although H5N1 has been reported to cause disseminated infection in humans, the lungs remain the main site of viral replication. While IgG transudates from plasma to the lungs to mediate protection after intravenous administration, very high dosages are required to effectively eliminate nasal viral shedding. To improve recovery of IgG at the lungs, vectored delivery directly at the nasopharyngeal mucosa has been suggested as a practical strategy. This approach has yielded encouraging results in mouse and ferret models of H5N1 infection, with the added advantage of antibody expression lasting up to 100 days (Limberis et al., 2013).

In this study, xi-IgA₁-9F4 was generated as this isotype is predominant in the nasal mucosa during influenza infection and the presence of specific secretory IgA in the upper respiratory tract is associated with resistance to severe respiratory disease (Weltzin and Monath, 1999). Thus far, only one IgA₁, generated using mouse hybridoma (Ye et al., 2010), has been reported. IgA potentially offers significant advantages over IgG. Firstly, IgA does not fix complement via the classical pathway (Woof and Russell, 2011) and is therefore believed to be less pro-inflammatory than IgG MAbs. This feature could be particularly important for H5N1 infection, where disease severity correlates with exacerbated inflammation. Secondly, IgA permits intranasal administration (Ye et al., 2010), allowing IgA to neutralize influenza A virus at the primary site of infection, thereby preventing colonization and invasion of host cells. Alternatively, dimeric IgA can be generated for systemic administration, allowing IgA to bind to polymeric IgG receptors (plgR) located at the basal membrane of epithelial cells for transepithelial transport to the mucus layer (Tamura et al., 2005). Both routes of administration enable IgA access to the upper respiratory tract, where inhibition of viral replication can occur. In contrast, IgG MAb activity is localized in the lung. Interestingly, it was recently shown that IgA₁, but not IgG₁, prevents transmission of influenza viruses in guinea pig model (Seibert et al., 2013).

Dimeric IgA will also encounter intracellular virus present within endosomes during transepithelial transport (Tamura et al., 2005). MAbs that prevent the fusion process can therefore bind to virus present within the endosomes and interfere with virus uncoating during entry. The polymerization of IgA also enhances its antiviral immune responses due to the increased ability for antigen agglutination. Polymeric IgA variants of originally IgG antibodies have been shown to improve antibody reactivity to specific antigen for other diseases affecting mucosal tissues (Liu et al., 2003), thus, the generation of polymeric xi-IgA1-9F4 is a possible future direction in improving its neutralizing potency.

Chimeric MAb xi-IgG₁-9F4 and xi-IgA₁-9F4 are conformational dependent antibodies with three epitope sites contributing to binding to HA1. Using a combination of deletion and substitution mutants, ⁶⁰WLL⁶² (SEQ ID NO: 19), ⁷⁵EWSYIV⁸⁰ (SEQ ID NO: 20) and ²⁶⁰I/LVKK²⁶³ (SEQ ID NOs: 17 and 18) (based on mature H5 numbering) were found to be critical for antibody binding to the non-RBD vestigial esterase domain. These three epitopes are well conserved among all human H5 sequences deposited in The Influenza Research Database (www.fludb.org). To our knowledge, these epitope sites are unique to xi-IgG₁-9F4, xi-IgA₁-9F4 and related 9F4 and have not been described for other anti-H5 MAbs. The only other anti-H5 MAb described that binds near this locality but with different critical residues is H5M9 (key residues: D53, Y274, E83, and N276) (Zhu et al. 2013). The low occurrence of antibodies targeting this region suggests their rarity in the immune repertoire. One possible reason attributing to such immune sub-dominance could be that this region is not easily accessible within the homotrimeric structure of HA. The epitope site ⁶⁰WLL⁶² is not readily surface exposed (data not shown). It is known that at low pH, HA1 dissociates from HA2, however, there is no available structural information on the position of HA1 within the fusiongenic intermediates. It is likely that ⁶⁰WLL⁶² becomes more exposed during the transition from pre-fusion to post-fusion forms and the association of the antibody traps H5 in these intermediate conformations thereby preventing fusion.

Notably, binding of the hereinbefore described antibodies is independent of HA glycosylation, indicating that the neutralizing activity of MAb xi-IgG₁-9F4 and xi-IgA₁-9F4 may be resilient against drift variants with differing glycosylation patterns.

In summary, xi-IgG₁-9F4 and xi-IgA₁-9F4 are conformation dependent neutralizing MAb which display heterologous protection against multiple clades of HPAI H5N1. The novelty of these antibodies and the conformational epitope to which they specifically bind suggests that these MAbs could be suitable in combination approaches with RBD- or HA2-targeting MAbs in a polyclonal passive immunotherapeutic fashion.

### REFERENCES

Cao, Z., Meng, J., Li, X., Wu, R., Huang, Y. and He, Y. (2012) The Epitope and Neutralization Mechanism of AVFIulgG01, a Broad-Reactive Human Monoclonal Antibody against H5N1 Influenza Virus. PLoS One 7(5), 25.
Chao, D. L., Bloom, J. D., Kochin, B. F., Antia, R. and Longini, I. M., Jr. (2012) The global spread of drug-resistant influenza. J R Soc Interface 9(69), 648-56.
Du, L., Jin, L., Zhao, G., Sun, S., Li, J., Yu, H., Li, Y., Zheng, B. J., Liddington, R. C., Zhou, Y. and Jiang, S. (2013) Identification and structural characterization of a broadly neutralizing antibody targeting a novel conserved epitope on the influenza virus H5N1 hemagglutinin. J Virol 87(4), 2215-25.
Ehrenmann, F., Kaas, Q. and Lefranc, M. P. (2010) IMGT/3Dstructure-DB and IMGT/DomainGapAlign: a database and a tool for immunoglobulins or antibodies, T cell receptors, MHC, IgSF and MhcSF. Nucleic Acids Res 38(Database issue), 9.
Frey, A., Di Canzio, J. and Zurakowski, D. (1998) A statistically defined endpoint titer determination method for immunoassays. J Immunol Methods 221(1-2), 35-41.
Garcia, J. M. and Lai, J. C. (2011) Production of influenza pseudotyped lentiviral particles and their use in influenza research and diagnosis: an update. Expert Rev Anti Infect Ther 9(4), 443-55.
Le, Q. M., Kiso, M., Someya, K., Sakai, Y. T., Nguyen, T. H., Nguyen, K. H., Pham, N. D., Ngyen, H. H., Yamada, S., Muramoto, Y., Horimoto, T., Takada, A., Goto, H., Suzuki, T., Suzuki, Y. and Kawaoka, Y. (2005) Avian flu: isolation of drug-resistant H5N1 virus. Nature 437(7062).
Limberis, M. P., Adam, V. S., Wong, G., Gren, J., Kobasa, D., Ross, T. M., Kobinger, G. P., Tretiakova, A. and Wilson, J. M. (2013) Intranasal antibody gene transfer in mice and ferrets elicits broad protection against pandemic influenza. Science translational medicine 5(187), 3006299.
Liu, F., Bergami, P. L., Duval, M., Kuhrt, D., Posner, M. and Cavacini, L. (2003) Expression and functional activity of isotype and subclass switched human monoclonal antibody reactive with the base of the V3 loop of HIV-1 gp120. AIDS Res Hum Retroviruses 19(7), 597-607.
Meng, W., Pan, W., Zhang, A. J., Li, Z., Wei, G., Feng, L., Dong, Z., Li, C., Hu, X., Sun, C., Luo, Q., Yuen, K. Y., Zhong, N. and Chen, L. (2013) Rapid Generation of Human-Like Neutralizing Monoclonal Antibodies in Urgent Preparedness for Influenza Pandemics and Virulent Infectious Diseases. PLoS One 8(6).
Nason, E. L., Wetzel, J. D., Mukherjee, S. K., Barton, E. S., Prasad, B. V. and Dermody, T. S. (2001) A monoclonal antibody specific for reovirus outer-capsid protein sigma3 inhibits sigma1-mediated hemagglutination by steric hindrance. J Virol 75(14), 6625-34.
Oh, H. L., Akerstrom, S., Shen, S., Bereczky, S., Karlberg, H., Klingstrom, J., Lal, S. K., Mirazimi, A. and Tan, Y. J. (2010) An antibody against a novel and conserved epitope in the hemagglutinin 1 subunit neutralizes numerous H5N1 influenza viruses. J Virol 84(16), 8275-86.
Prabakaran, M., Prabhu, N., He, F., Hongliang, Q., Ho, H. T., Qiang, J., Meng, T., Goutama, M. and Kwang, J. (2009) Combination therapy using chimeric monoclonal antibodies protects mice from lethal H5N1 infection and prevents formation of escape mutants. PLoS One 4(5).
Seibert, C. W., Rahmat, S., Krause, J. C., Eggink, D., Albrecht, R. A., Goff, P. H., Krammer, F., Duty, J. A., Bouvier, N. M., Garcia-Sastre, A. and Palese, P. (2013) Recombinant IgA is sufficient to prevent influenza virus transmission in guinea pigs. J Virol 87(14), 7793-804.
Shen, S., Mahadevappa, G., Oh, H. L., Wee, B. Y., Choi, Y. W., Hwang, L. A., Lim, S. G., Hong, W., Lal, S. K. and Tan, Y. J. (2008) Comparing the antibody responses against recombinant hemagglutinin proteins of avian influenza A (H5N1) virus expressed in insect cells and bacteria. J Med Virol 80(11), 1972-83.
Tamura, S., Tanimoto, T. and Kurata, T. (2005) Mechanisms of broad cross-protection provided by influenza virus infection and their application to vaccines. Jpn J Infect Dis 58(4), 195-207.
Tang, J. W., Ngai, K. L., Wong, J. C., Lam, W. Y. and Chan, P. K. (2008) Emergence of adamantane-resistant influenza A(H3N2) viruses in Hong Kong between 1997 and 2006. J Med Virol 80(5), 895-901.
Ter Meulen, J., van den Brink, E. N., Poon, L. L., Marissen, W. E., Leung, C. S., Cox, F., Cheung, C. Y., Bakker, A. Q., Bogaards, J. A., van Deventer, E., Preiser, W., Doerr, H. W., Chow, V. T., de Kruif, J., Peiris, J. S. and Goudsmit, J. (2006) Human monoclonal antibody combination against SARS coronavirus: synergy and coverage of escape mutants. PLoS Med 3(7).
Underwood, P. A. (1982) Mapping of antigenic changes in the haemagglutinin of Hong Kong influenza (H3N2) strains using a large panel of monoclonal antibodies. J Gen Virol 62(Pt 1), 153-69.
Velkov, T., Ong, C., Baker, M. A., Kim, H., Li, J., Nation, R. L., Huang, J. X., Cooper, M. A. and Rockman, S. (2013) The antigenic architecture of the hemagglutinin of influenza H5N1 viruses. Mol Immunol 56(4), 705-19.
Weltzin, R. and Monath, T. P. (1999) Intranasal antibody prophylaxis for protection against viral disease. Clin Microbiol Rev 12(3), 383-93.
Wiley, D. C., Wilson, I. A. and Skehel, J. J. (1981) Structural identification of the antibody-binding sites of Hong Kong influenza haemagglutinin and their involvement in antigenic variation. Nature 289(5796), 373-8.
Woof, J. M. and Russell, M. W. (2011) Structure and function relationships in IgA. Mucosal Immunol 4(6), 590-7.
Ye, J., Shao, H., Hickman, D., Angel, M., Xu, K., Cai, Y., Song, H., Fouchier, R. A., Qin, A. and Perez, D. R. (2010) Intranasal delivery of an IgA monoclonal antibody effective against sublethal H5N1 influenza virus infection in mice. Clin Vaccine Immunol 17(9), 1363-70.
Ye, J., Shao, H. and Perez, D. R. (2012) Passive immune neutralization strategies for prevention and control of influenza A infections. Immunotherapy 4(2), 175-86.
Zuo, T., Shi, X., Liu, Z., Guo, L., Zhao, Q., Guan, T., Pan, X., Jia, N., Cao, W., Zhou, B., Goldin, M. and Zhang, L. (2011) Comprehensive analysis of pathogen-specific antibody response in vivo based on an antigen library displayed on surface of yeast. J Biol Chem 286(38), 33511-9.

1. An isolated chimeric antibody, variant, mutant or fragment thereof, wherein the antibody, variant, mutant or fragment thereof is capable of specifically binding to a conformational (non-linear) epitope of influenza A virus subtype H5N1, wherein the conformational epitope comprises the amino acid sequence I/LVKK and the amino acid sequence WLL.

2. The chimeric antibody of claim 1, wherein the conformational epitope comprises three antigenic sites, wherein a first site comprises the amino acid sequence I/LVKK, a second site comprises the amino acid sequence WLL and the third site comprises the amino acid sequence EWSYIV.

3. The chimeric antibody of claim 1, wherein the antibody, variant, mutant or fragment thereof is a mouse-human chimeric antibody.

4. The chimeric antibody of claim 1, wherein the antibody comprises at least one variable heavy chain and at least one variable light chain, wherein the heavy chain comprises the VH domain of mouse monoclonal antibody 9F4, a variant, mutant or fragment thereof and the light chain comprises the VL domain of mouse monoclonal antibody 9F4, a variant, mutant or fragment thereof.

5. The chimeric antibody of claim 4, wherein the antibody comprises the mouse VH domain ligated to human IgG₁ heavy chain constant (CH) domain and the mouse VL domain ligated to human light chain kappa constant (CL) domain; or wherein the antibody comprises the mouse VH domain ligated to human IgA₁ heavy chain constant (CH) domain and the mouse VL domain ligated to human light chain kappa constant (CL) domain.

6. The chimeric antibody of claim 4, comprising (a) a variable heavy chain comprising
the amino acid sequence of SEQ ID NO: 1, a variant, mutant or fragment thereof, and a variable light chain comprising the amino acid sequence of SEQ ID NO: 2, a variant, mutant or fragment thereof, for mouse-human chimeric IgG₁ antibody, or
(b) a variable heavy chain comprising the amino acid sequence of SEQ ID NO: 1, a variant, mutant or fragment thereof, and a variable light chain comprising the amino acid sequence of SEQ ID NO: 2, a variant, mutant or fragment thereof, for mouse-human chimeric IgA₁ antibody.

7. The chimeric antibody of claim 6, wherein in (a) the variable heavy chain sequence of SEQ ID NO: 1 is encoded by the nucleic acid sequence of SEQ ID NO: 3 and the variable light chain SEQ ID NO: 2 is encoded by the nucleotide sequence of SEQ ID NO: 4, and wherein in (b) the variable heavy chain sequence of SEQ ID NO: 5 is encoded by the nucleic acid sequence of SEQ ID NO: 7 and the variable light chain SEQ ID NO: 6 is encoded by the nucleotide sequence of SEQ ID NO: 8.

8. The chimeric antibody of claim 1, wherein the antibody has the H5N1 binding and neutralization characteristics of mouse monoclonal antibody 9F4, xi-IgG₁-9F4 or xi-IGA₁-9F4.

9. The chimeric antibody of claim 1, wherein the antibody binds to clade 2.3.4 H5 HA.

10. The chimeric antibody of claim 1, wherein the antibody is linked with at least one drug, preferably an anti-viral drug.

11. A method of producing at least one mouse-human chimeric antibody which binds influenza A virus subtype H5N1, the method comprising the steps of:
a. Ligating a 9F4 VH domain nucleic acid encoding SEQ ID NO: 1 to a human IgG₁ CH domain and ligating a 9F4 VL domain nucleic acid encoding SEQ ID NO: 2 to a human CL domain in a single IgG₁ constant region expression vector; or
b. Ligating a 9F4 VH domain nucleic acid encoding SEQ ID NO: 5 to human IgA₁ CH domain in a first cloning vector, and ligating a 9F4 VL domain nucleic acid encoding SEQ ID NO: 6 to a human CL domain in a second cloning vector;
c. Transfecting the resulting chimeric construct or constructs into a suitable cell line; and
d. Collecting cell culture supernatants and extracting and purifying the chimeric antibody.

12-13. (canceled)

14. A method of treatment of influenza A subtype H5N1 disease, the method comprising administering to a subject in need thereof an efficacious amount of at least one chimeric antibody, variant, mutant or a fragment thereof as defined in claim 1.

15. The method of claim 14, wherein the at least one antibody, variant, mutant or a fragment thereof is administered in combination with one or more other antibodies directed to Influenza A which bind to virus epitopes that do not compete with binding of same.

16-18. (canceled)

19. An isolated nucleic acid molecule encoding:
(a) at least one variable heavy chain of the chimeric antibody or a fragment thereof as defined in claim 1, wherein the heavy chain comprises the amino acid sequence of SEQ ID NO: 1, a variant, mutant or fragment thereof; and at least one variable light chain of the antibody, variant, mutant or a fragment thereof as defined in claim 1, wherein the light chain comprises the amino acid sequence of SEQ ID NO: 2, a variant, mutant or fragment thereof, or
(b) at least one variable heavy chain of the antibody, variant, mutant or a fragment thereof as defined in claim 1, wherein the heavy chain comprises the amino acid sequence of SEQ ID NO: 5, a variant, mutant or fragment thereof; and at least one variable light chain of the antibody, variant, mutant or a fragment thereof as defined in claim 1, wherein the light chain comprises the amino acid sequence of SEQ ID NO: 6, a variant, mutant or fragment thereof, and
wherein the nucleic acid molecule encodes an IgG₁ or an IgA₁ chimeric antibody, respectively.

20. The isolated nucleic acid molecule of claim 19, wherein in a) the variable heavy chain nucleic acid sequence has at least 90% sequence identity to SEQ ID NO: 3 and the variable light chain nucleic acid sequence has at least 90% sequence identity to SEQ ID NO: 4; and in b) the variable heavy chain nucleic acid sequence has at least 90% sequence identity to SEQ ID NO: 7 and the variable light chain nucleic acid sequence has at least 90% sequence identity to SEQ ID NO: 8.

21. The isolated nucleic acid molecule of claim 20, wherein in a) the variable heavy chain nucleic acid sequence is SEQ ID NO: 3 and the variable light chain nucleic acid sequence is SEQ ID NO: 4; and in b) the variable heavy chain nucleic acid sequence is SEQ ID NO: 7 and the variable light chain nucleic acid sequence is SEQ ID NO: 8.

22-23. (canceled)

24. An expression vector comprising the nucleic acid molecule defined in claim 14.


SEQUENCE LISTING (21 sequences)

SEQ ID NO: 1 - protein, 113 aa, Artificial Sequence
  aa VH sequence in xi-IgG-9F4
QQSEAELARPGASVKMSCKASGFTLTTFTIHWVKQRPGQGLEWIGYINPRSGYTDYNQKFKDNTTLTVDKSSSTAYMQLSSLTSEDSAVFYCARSYYDYDVFDYWGQGTTLTV

SEQ ID NO: 2 - protein, 102 aa, Artificial Sequence
  aa VL seq in xi-IgG-9F4
DVQMTQSPSSLSASLGGKVTITCTARQDINKYIAWYQHKPGKGPRLLIHYTSTLQPGIPSRFSGSGSGTDYSFTISNLEPEDIATYYCLQYDNLVTFGGGTK

SEQ ID NO: 3 - DNA, 339 bp, Artificial Sequence
  nuc VH seq in xi-IgG-9F4
cagcagtctgaagctgaactggcaagacctggggcctcagtgaagatgtcctgcaaggcttctggcttcaccttgactaccttcacgatccactgggtaaaacagaggcctggacagggtctggaatggattggatacattaatcctcgcagtggatatactgactacaatcagaagttcaaggacaataccacattgactgtagacaaatcctccagcacagcctacatgcaactgagcagcctgacatctgaggactctgcggtcttttactgtgcaagatcctactatgattacgacgtctttgactactggggccaaggcaccactctcacagtc

SEQ ID NO: 4 - DNA, 306 bp, Artificial Sequence
  nuc VL seq in xi-IgG-9F4
gacgtccagatgacacagtctccatcctcactgtctgcatctctgggaggcaaagtcaccatcacttgcacggcaaggcaagacattaacaagtatatcgcttggtaccaacacaagcctggaaaaggtcctaggctgctcatacattacacatctacattgcagccaggcatcccatcaaggttcagtggaagtgggtctgggacagattattctttcaccatcagcaacctggagcctgaagatattgcaacttattattgtctacagtatgataatctggtcacgttcggtggtgggaccaaa

SEQ ID NO: 5 - protein, 119 aa, Artificial Sequence
  aa VH sequence in xi-IgA-9F4
QVQLQQSEAELARPGASVKMSCKASGFTLTTFTIHWVKQRPGQGLEWIGYINPRSGYTDYNQKFKDNTTLTVDKSSSTAYMQLSSLTSEDSAVFYCARSYYDYDVFDYWGQGTTLTVSS

SEQ ID NO: 6 - protein, 107 aa, Artificial Sequence
  aa VL seq in xi-IgA-9F4
DVQMTQSPSSLSASLGGKVTITCTARQDINKYIAWYQHKPGKGPRLLIHYTSTLQPGIPSRFSGSGSGTDYSFTISNLEPEDIATYYCLQYDNLVTFGGGTKLELKR

SEQ ID NO: 7 - DNA, 357 bp, Artificial Sequence
  nuc VH seq in xi-IgA-9F4
caggtccagctgcagcagtctgaagctgaactggcaagacctggggcctcagtgaagatgtcctgcaaggcttctggcttcaccttgactaccttcacgatccactgggtaaaacagaggcctggacagggtctggaatggattggatacattaatcctcgcagtggatatactgactacaatcagaagttcaaggacaataccacattgactgtagacaaatcctccagcacagcctacatgcaactgagcagcctgacatctgaggactctgcggtcttttactgtgcaagatcctactatgattacgacgtctttgactactggggccaaggcaccactctcacagtctcctca

SEQ ID NO: 8 - DNA, 321 bp, Artificial Sequence
  nuc VL seq in xi-IgA-9F4
gacgtccagatgacacagtctccatcctcactgtctgcatctctgggaggcaaagtcaccatcacttgcacggcaaggcaagacattaacaagtatatcgcttggtaccaacacaagcctggaaaaggtcctaggctgctcatacattacacatctacattgcagccaggcatcccatcaaggttcagtggaagtgggtctgggacagattattctttcaccatcagcaacctggagcctgaagatattgcaacttattattgtctacagtatgataatctggtcacgttcggtggtgggaccaaactggagctgaaacgg

SEQ ID NO: 9 - DNA, 35 bp, Artificial Sequence
  nuc VH primer Forward
tagcccaggtgcaattgcagcagtctgaagctgaa

SEQ ID NO: 10 - DNA, 33 bp, Artificial Sequence
  nuc VH primer Reverse
ccgccctctcgagactgtgacagtggtgccttg

SEQ ID NO: 11 - DNA, 34 bp, Artificial Sequence
  nuc VL primer Forward
gatcgaagtgcactccgacgtccagatgacacag

SEQ ID NO: 12 - DNA, 33 bp, Artificial Sequence
  nuc VL primer Reverse
ccgtttgatctgcagtttggtcccaccaccgaa

SEQ ID NO: 13 - DNA, 23 bp, Artificial Sequence
  nuc VH primer Forward
cggaattcgcaggtccagctgca

SEQ ID NO: 14 - DNA, 25 bp, Artificial Sequence
  nuc VH primer Reverse
ctagctagctgaggagactgtgaga

SEQ ID NO: 15 - DNA, 27 bp, Artificial Sequence
  nuc VL primer Forward
cggaattcagacgtccagatgacacag

SEQ ID NO: 16 - DNA, 25 bp, Artificial Sequence
  nuc VL primer Reverse
gaacgtacgccgtttcagctccagt

SEQ ID NO: 17 - protein, 4 aa, Artificial Sequence
  Amino acid sequence
IVKK

SEQ ID NO: 18 - protein, 4 aa, Artificial Sequence
  Amino acid sequence
LVKK

SEQ ID NO: 19 - protein, 3 aa, Artificial Sequence
  Amino acid sequence
WLL

SEQ ID NO: 20 - protein, 6 aa, Artificial Sequence
  Amino acid sequence
EWSYIV

SEQ ID NO: 21 - protein, 11 aa, Artificial Sequence
  Amino acid sequence
KIVKKGDSYIM